(19) United States
(12) Patent Application Publication
(10) Pub. No.: US 2024/0226173 A1
(43) Pub. Date: Jul. 11, 2024

(54) METHODS AND COMPOSITIONS FOR TREATING LIVER DISEASE

(71) Applicant: PrimeGen US, Inc., Santa Ana, CA (US)

(72) Inventor: Joel Marh, Santa Ana, CA (US)

(21) Appl. No.: 18/560,304

(22) PCT Filed: May 12, 2022
(86) PCT No.: PCT/US2022/028948

§ 371 Date: Nov. 10, 2023

Related U.S. Application Data
(60) Provisional application No. 63/188,121, filed on May 13, 2021.

(51) Int. Cl. A61K 35/28; A61P 1/16; C12N 5/0775

(52) U.S. Cl. CPC A61K 35/28; A61P 1/16; C12N 5/0665

(57) ABSTRACT

Provided are methods of treatment comprising administering to a subject, in need thereof, a therapeutically effective amount of activated stem cells to the affected tissue or organ. The methods described herein are treatment modalities employing mesenchymal stem cells (MSC) in the treatment of mammals, as well as MSC purification and formulation methods including the “activation” or “preconditioning” of stem cells.

### TECHNICAL FIELD

This disclosure relates to methods and compositions for treating liver disease using stem cells. More specifically described herein are treatment modalities employing mesenchymal stem cells (MSC) in the treatment of mammals, as well as MSC purification and formulation methods including the “activation” or “preconditioning” of stem cells.

### BACKGROUND

Stem cells are specialized cells, capable of renewing themselves through cell division as well as differentiating into multi-lineage cells. These cells are categorized as embryonic stem cells (ESC), induced pluripotent stem cells (iPSC), and adult stem cells. Mesenchymal stem cells (MSC) are adult stem cells which can be isolated from human and animal sources. Human MSC (hMSC or huMSC) are non-haematopoietic, multipotent stem cells with the capacity to differentiate into mesodermal lineage such as osteocytes, adipocytes and chondrocytes as well ectodermal (neurocytes) and endodermal lineages (hepatocytes). MSC express cell surface markers including cluster of differentiation (CD)29, CD44, CD73, CD90, CD105, and lack the expression of CD14, CD34, CD45, and HLA (human leucocyte antigen)-DR. hMSC have been isolated from various tissues, including adipose tissue, amniotic fluid, endometrium, dental tissues, umbilical cord, and Wharton's jelly. hMSC have been cultured long-term in specific media without any severe abnormalities.

MSC display immunomodulatory features, and can secrete cytokines and immune-receptors which regulate the microenvironment in the host tissue. Multilineage potential, immunomodulation and secretion of anti-inflammatory molecules make MSC an effective tool in the treatment of chronic diseases.

### SUMMARY

The present disclosure is based, at least in part, on the non-limiting theory that MSC can be used to treat various conditions, for example conditions afflicting mammals such as liver disease, by utilizing the MSC to produce factors beneficial in the treatment of liver disease. Such factors can include cytokines, for example IL-6. IL-6 is a pleiotropic cytokine, exerting a variety of effects on inflammation, liver regeneration, and defense against infections by regulating adaptive immunity. Due to its high abundance in inflammatory settings, IL-6 is often viewed as a detrimental cytokine. However, accumulating evidence supports the view that IL-6 has a beneficial impact in numerous liver pathologies, due to its roles in liver regeneration and in promoting an anti-inflammatory response in certain conditions. IL-6 promotes proliferation, angiogenesis and metabolism, and downregulates apoptosis and oxidative stress; together these functions are critical for mediating hepatoprotection. IL-6 is also an important regulator of adaptive immunity where it induces T cell differentiation and regulates autoimmunity. It can augment antiviral adaptive immune responses and mitigate exhaustion of T cells during chronic infection.

Disclosed embodiments comprise compositions for treating a patient, for example a human or non-human mammal, suffering from liver disease or symptoms thereof, said compositions comprising MSC derived from progenitor cells isolated from, for example, adipose tissue, umbilical cord, placental tissue, bone marrow, dental tissue, testicle tissue, uterine tissue, umbilical cord tissue, or skin tissue, that are allogeneic or autologous to a target patient; and a saline solution, wherein the composition can prevent, reduce, or eliminate the symptoms of liver disease in a target patient.

Disclosed embodiments comprise therapeutic use of “activated” MSC. For example, embodiments comprise purifying MSC with different abilities to maximize their therapeutic benefit for specific use, for example using cell-sorting procedures such as Magnetic-Activated Cell Sorting (MACS) or Fluorescence-Activated Cell Sorting (FACS). Further embodiments comprise activating MSC with specific stimulatory agents including, for example, cytokines, reactive proteins, chemicals, small molecules, and combinations thereof. These stimulatory agents can enhance or suppress MSC function; for example, immunosuppression by MSC can be induced by proinflammatory cytokines.

Disclosed embodiments comprise MSC that have been frozen and thawed. The MSC can comprise unactivated or activated MSC. In embodiments comprising the use of activated then frozen MSC, the MSC can be activated again. In embodiments comprising the use of activated then frozen MSC, the activated MSC do not require additional activation.

Further embodiments comprise the use of MSC in combination treatments, for example the use of MSC in combination with a drug or pharmaceutical active agent or pharmaceutical composition. For example, disclosed embodiments comprise administration of MSC in combination with, for example, exosomes, such as purified exosomes.

### BRIEF DESCRIPTION OF THE DRAWINGS

FIG. 1 shows MSC treatment rescued mortality of humanized FRG mice with alcoholic hepatitis (Overall survival). Alcohol binging Cohort shows a higher survival rate in MSC treated mice compared to PBS control group. Log-rank (Mantel-Cox) test P<0.0001. Gehan-Breslow-Wilcoxon test P<0.0001.

FIG. 2 depicts ALT and AST from Cohort 1; ALT and AST were measured by biochemical assays. Both AST and ALT levels were reduced after non-activated MSC repetitive injection into mice with alcoholic hepatitis. Student's T-test analyses demonstrated that post-treatment ALT and AST showed significantly lower values in MSC repetitive injection groups while PBS injection did not significantly alter the ALT and AST values.

FIG. 3 depicts activated MSC treatment rescued mortality of humanized FRG mice with alcoholic hepatitis (Overall Survival); FRG mouse survival data in presence/absence of MSC injection are shown. Log-rank (Mantel-Cox) test P value was 0.0130, log-rank test for trend P value was 0.0032. Gehan-Breslow-Wilcoxon test P value was 0.0270.

FIG. 4 depicts histological findings at death or sacrifice of humanized FRG mice with alcoholic hepatitis;

FIG. 4A. PBS control mice showing mild (1+) steatosis, HE×100×.

FIG. 4B. PBS control mice showing mild (1+ steatosis), HE×100×.

FIG. 4C. Mice treated with Non activated IP showing mild (1+ steatosis) and 30% necrosis, HE×100×.

FIG. 4D. Mice treated with Non activated MSC IP showing no steatosis, HE×100×.

FIG. 4E. Mice treated with activated MSC IP showing no significant steatosis (<5%), HE×100×.

FIG. 4F. Mice treated with activated MSC IV showing mild (1+ steatosis) and 20% necrosis, HE×100×

FIG. 5 shows AST and ALT levels from Cohort 2; ASL and ALT were drawn at onset of treatment and at death including those that were euthanized. Student's T-test analyses showed that post-treatment ALT and AST showed significantly lower values in MSC repetitive injection groups, while PBS injection did not significantly alter the ALT and AST values in mouse serum.

FIG. 6 shows cytokine expression by MSC after MSC activation.

FIG. 7 shows baseline chem panel data (pig).

FIG. 8 shows baseline chem panel data (pig).

FIG. 9 shows endpoint chem panel data for the pig of FIGS. 7 and 8.

FIG. 10 shows endpoint chem panel data for the pig of FIGS. 7 and 8.

FIG. 11 shows baseline chem panel data (pig).

FIG. 12 shows baseline chem panel data (pig).

FIG. 13 shows endpoint chem panel data for the pig of FIGS. 11 and 12.

FIG. 14 shows endpoint chem panel data for the pig of FIGS. 11 and 12.

FIG. 15 shows baseline chem panel data (pig).

FIG. 16 shows baseline chem panel data (pig).

FIG. 17 shows endpoint chem panel data for the pig of FIGS. 15 and 16.

FIG. 18 shows endpoint chem panel data for the pig of FIGS. 15 and 16.

FIG. 19 shows Complete Blood Count baseline data demonstrating the health of the test animals.

FIG. 20 shows cells harvested per flask as described in Example 8.

FIG. 21 shows cell population doubling time after 48-hour culture as described in Example 6.

FIG. 22 shows the average viability of activated cells from Example 8.

FIG. 23 shows that the “Original” P.5 cells (Example 8) released a higher concentration per cell. If those previously activated cells were frozen and thawed and cultured for 48 hrs, the cells were still able to produce the important cytokines at a high level (such cytokines as IL6, MCP1, TGFB). Also the data from the graph also shows if those previously activated cells were frozen and thawed and cultured for 48 hours and reactivated a second time. The cells were still able to produce the important cytokines as the cells that were already activated one time or the cells that were already activated and frozen/thawed and cultured for 48 hours.

FIG. 24 shows that Mesenchymal stem cell (MSC) treatment rescued mortality of humanized Fah−/−, Rag2−/−, and Il2rgc−/− (FRG) mice with alcoholic hepatitis.

FIG. 24A schematic illustration showing relevant time points for ASH1 cohort.

FIG. 24B mice treated with nonactivated MSCs had significantly better survival that mice treated with phosphate-buffered saline (PBS) (Wilcoxon p<0.0001). HSC, hematopoietic stem cell; Hep, Hepatocytes; IP, intraperitoneally; IV, intravenously; qRT-PCR, quantitative real-time polymerase chain reaction.

FIG. 25 shows MSC treatment rescued mortality of humanized FRG mice with alcoholic hepatitis.

FIG. 25A schematic showing relevant time points for ASH2 cohort.

FIG. 25B mice treated with activated MSCs via any route had significantly better survival than mice treated with nonactivated MSCs or PBS alone (Wilxcoxon p<0.0001). Group activated MSC IP+IV, activated MSC IV, activated MSC IP, and activated MSC IP+IV overlapped, and all had the same 100% survival line. HFCD, high-fat chow diet. *=p<0.05.

FIG. 26 shows aspartate aminotransferase (AST) and alanine aminotransferase (ALT) levels from cohort 2. AST and ALT were drawn at onset of treatment and at death, including those that were killed.

FIG. 26A Post hoc analysis with Bonferroni adjustment for last-day ALT: activated vs. PBS: <0.0001; nonactivated vs. PBS: <0.0001.

FIG. 26B Post hoc analysis with Bonferroni adjustment for last-day AST: activated vs. PBS<0.0001: nonactivated vs. PBS: <0.0001. Post hoc analysis with Bonferroni adjustment for change of AST: activated vs. PBS: <0.0001; nonactivated vs. PBS: <0.0001.

FIG. 27 shows that vimentin validates the location of human MSCs in the liver.

FIG. 27A Vimentin (human-specific) immunohistochemistry (IHC) shows minimal expression in only the activated MSC-treated group. Scale bar=10 μm.

FIG. 27B Quantitative PCR shows a significant 2-fold increase in Vimentin MSC marker compared with PBS control group.

FIG. 27C Ki-67 marker was significantly elevated in activated MSCs compared with PBS and nonactivated MSCs.

FIG. 27D myeloperoxidase (MPO) expression marker was significantly decreased in both activated and nonactivated MSCs compared with PBS-treated group.

FIG. 27E Activated MSCs expressed significantly lower human serum albumin levels compared with PBS control. DAPI, 4′,6-diamidino- 2-phenylindole. *=p<0.05, **: P<0.01, ****: p<0.0001.

FIG. 28 shows receptor-interacting protein kinase (RIPK3) IHC provides insights about the necroptosis pathway.

FIG. 28A confocal fluorescent images showing DAPI (blue) and RIPK3 (red) expression in PBS, nonactivated MSCs, and activated MSCs. Scale bar=10 μm.

FIG. 28B immunoreactive score (IRS) reveals significant decrease in RIPK3 expression in activated MSC tissue. Three representative paraffin-embedded liver tissues were stained for each group.

FIG. 28C Western blot showing RIP3 expression in activated MSC-treated and PBS-treated mice groups. RIP3 expression is decreased in the activated MSC group compared with the PBS control group.

FIG. 28D Western blot showing expression of B cell lymphoma 2 (BCL-2). Activated MSC-treated group shows highest expression of BCL-2 compared with PBS and nonactivated MSC groups.

FIG. 28E BCL-2 promoter is regulated by signal transducer and activator of transcription 3 (Stat3) and cyclic adenosine monophosphate response element-binding protein (CREB1) binding sites.

FIG. 28F Western blot showing a reduced cleaved Gasdermin D (GSDMD) expression in activated MSC-treated mice. Cleaved GSDMD was highly expressed in both nonactivated and PBS-treated mice.

FIG. 28G proposed hypothetical mechanism. *: p<0.05, **: P=<0.01, ***: p<0.001.

FIG. 29 shows Bioluminescence imaging shows a reduction of MSC after short hairpin CD44 (sh-CD44) transduction.

FIG. 29A images of mice after sh-scrambled activated MSC IP injection.

FIG. 29B images of mice after sh-CD44 transduced activated MSCs. Images show lower amount of Luciferase compared with sh-scrambled. ROI, region of interest.

FIG. 30 shows overall survival based on gender. Kaplan-Maier Plot shows minimal difference in survival between male and female mice.

FIG. 31 shows human mitochondria staining showing FRG substitution Rate. IHC of human mitochondria DNA demonstrated that humanized FRG mice have 60-70% substitution rate.

FIG. 32 shows an Elisa assay which reveals the importance of IL-6, IL-10, and MCP in Activated MSCs.

FIG. 33 shows that male mice had significantly better survival than female mice (p=0.03); however, these were equally represented between the groups.

FIG. 34 shows survival by treatment group; survival for mice dosed with 1 million cells was the only cohort found to have significantly better survival than the Placebo group (p=0.03).

FIG. 35 shows a comparison of liver histology of mice that died during each dose treatment group and mice that survived 28 days after treatment from each dose treatment group.

FIG. 35A shows placebo injected mouse #604 died showing Moderate (2+) steatosis, HE×100×.

FIG. 35B shows placebo injected mouse #606 survived 28 days after treatment showing Moderate (2+) steatosis, HE×100×.

FIG. 35C shows 28,000 activated MSC injected mouse #645 died showing Moderate (2+) steatosis, HE×100×.

FIG. 35D shows 28,000 activated MSC injected mouse #654 survived 28 days after treatment with Mild (1+) steatosis, HE×100×.

FIG. 35E shows 100,000 activated MSC injected mouse #658 died showing Moderate (2+) steatosis and necrosis, HE×100×.

FIG. 35F shows 100,000 activated MSC injected mouse #119 survived 28 days after treatment showing Mild (1+) steatosis, HE×100×.

FIG. 35G shows 250,000 activated MSC injected mouse #701 died showing Marked (3+) steatosis and necrosis, HE×100×.

FIG. 35H shows 250,000 activated MSC injected mouse #607 survived 28 days after treatment showing Mild (1+) steatosis, HE×100×.

FIG. 35I shows 500,000 activated MSC injected mouse #691 died showing Marked (3+) steatosis and necrosis, HE×100×.

FIG. 35J shows 500,000 activated MSC injected mouse #632 survived 28 days after treatment showing Mild (1+) steatosis, HE×100×.

FIG. 35K shows 1,000,000 activated MSC injected mouse #727 died showing Marked (3+) steatosis, HE×100×.

FIG. 35L shows 1,000,000 activated MSC injected mouse #601 survived 28 day after treatment with Mild (1+) steatosis, HE×100×.

### DETAILED DESCRIPTION

The liver is a vital organ located in the upper right-hand side of the abdomen. It weighs 2-3 pounds, and performs numerous functions in the body, including metabolizing and detoxifying toxic substances, converting food-derived nutrients, regulating blood clotting, maintaining hormone balances, storing vitamins, producing immune system components, and producing bile, which is essential for digestion.

Therefore, liver disease can severely impact quality of life. Causes may include infection, injury, exposure to drugs or toxic compounds, an autoimmune process, excessive drug or alcohol consumption, as well as others. Effects of liver disease can include inflammation, scarring, obstructions, blood clotting abnormalities, and liver failure.

The present disclosure is based, at least in part, on the benefits of treating patients using MSC, for example umbilical cord-derived, placental-derived or adipose-derived MSC. Treatments can include methods for ameliorating or lessening pain or other disease or condition symptoms, for example lessening at least one symptom of liver disease, for example lessening pain, nausea, fatigue, loss of appetite, yellowing of the skin, and combinations thereof. Subjects suitable for the disclosed treatments can include, for example, mammals, such as humans or animals. Treatments disclosed herein can comprise administration of other bioactive agents, for example an immunosuppressive agent.

Disclosed herein are methods of isolating and purifying MSC, for example umbilical cord MSC, placental MSC, adipose-derived MSC, and the like. Further embodiments comprise methods of propagating and banking MSC, for example umbilical cord-derived, placental-derived or adipose-derived MSC. Embodiments comprise purifying MSC based upon the cells' different abilities to maximize their therapeutic benefit for specific use, for example using cell-sorting procedures such as Magnetic-Activated Cell Sorting (MACS) or Fluorescence-Activated Cell Sorting (FACS).

Additional embodiments comprise activating MSC to modulate their therapeutic benefit, for example to increase their ability to suppress or enhance an immune response. In further embodiments, MSC can be activated prior to administration to a patient by contacting the MSC with at least one cytokine, for example, interferon gamma (IFNγ), Tumor Necrosis Factor alpha (TNFα), Interleukin-1 (IL-1), Interleukin-6 (IL-6), Interleukin-10 (IL-10), Interleukin-12 (IL-12), Interleukin-8 (IL-8), Macrophage Inflammatory Protein-1 beta (MIP-1b), or Interleukin-17 (IL-17).

### Definitions

ALD: Alcoholic Liver Disease.

ALT: Alanine aminotransferase.

AST: Aspartate aminotransferase.

HFCD: High-fat, high-cholesterol diet.

HSC: Human hematopoietic stem cell.

hUCMSC: human umbilical cord mesenchymal stem cells.

LPS: lipopolysaccharide.

MSC: Mesenchymal Stem Cells.

PBS: Phosphate-buffered saline.

“A” and “an” are used herein to refer to one or to more than one (i.e., to at least one) of the grammatical object of the article. By way of example, “an element” means one element or more than one element.

“Activate” (or “pre-condition”) as used herein in the context of MSC refers to the use of stimulatory agents including, for example, cytokines, reactive proteins, chemicals, small molecules, and combinations thereof to enhance an MSC function by contacting the MSC with the stimulatory agent.

“Comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

“Effective,” “effective amount,” and “therapeutically effective amount” refer to that amount of MSC or a pharmaceutical composition thereof that produces a beneficial result after administration.

“In vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

“Liver disease” or “hepatic disease” is any condition that causes liver inflammation or damage and may affect liver function.

“Or” as used herein should be understood to mean “and/or”, unless the context clearly indicates otherwise.

“Parenteral administration” and “administered parenterally” are art-recognized terms, and include modes of administration other than enteral and topical administration, such as injections, and include, without limitation, retro-orbital, intraocular, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

“Patient,” “subject,” or “host” to be treated by the subject method can mean either a human or non-human animal, such as a mammal, a fish, a bird, a reptile, or an amphibian.

“Pharmaceutically acceptable” or “therapeutically acceptable” refers to a substance which does not interfere with the effectiveness or the biological activity of the active ingredients and which is not toxic to a patient

“Pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Exemplary materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

“Pharmaceutical composition” refers to a formulation containing the therapeutically active agents described herein in a form suitable for administration to a subject. In embodiments, the pharmaceutical composition is in bulk or in unit dosage form. The quantity of active ingredient (e.g., MSC) in a unit dose of composition is an effective amount and can be varied according to the particular treatment involved. One skilled in the art will appreciate that it is sometimes necessary to make routine variations to the dosage depending on the age and condition of the patient. The dosage will also depend on the route of administration. In a preferred embodiment, the active ingredients are mixed under sterile conditions with a pharmaceutically acceptable carrier, and with any preservatives, buffers, or propellants that are required.

“Treatment” or “treating” refers to any therapeutic intervention in a mammal, for example a human or animal such as a companion animal, including: (i) prevention, that is, causing the clinical symptoms not to develop, e.g., preventing infection or inflammation from occurring and/or developing to a harmful state; (ii) inhibition, that is, arresting the development of clinical symptoms, e.g., stopping an ongoing infection so that the infection is eliminated completely or to the degree that it is no longer harmful; and/or (iii) relief, that is, causing the regression of clinical symptoms, e.g., causing a relief of fever and/or inflammation caused by or associated with a microbial infection. Treatment can comprise multiple administrations of compositions disclosed herein.

“Reducing”, “suppressing” and “inhibiting” have their commonly understood meaning of lessening or decreasing.

### Isolation of MSC

Disclosed embodiments can comprise methods of harvesting and isolating MSC.

In disclosed embodiments, MSC can be harvested and isolated from a variety of tissues, including, but not limited to, placenta, skeletal muscle, adipose tissue, umbilical cord, synovium, the circulatory system (e.g., blood), dental pulp, amniotic fluid, fetal blood, lung, liver, gonadal tissue, and bone marrow.

In embodiments, such methods can comprise aseptically collecting tissue from eligible mammalian donors. For example, in embodiments utilizing umbilical cord or placental MSC, tissue can be collected from full-term fetuses or during the third trimester of pregnancy. In embodiments, placenta is collected from specific pathogen-free donors, or from healthy donors with known health and travel history, free from adventitious agents. Multiple parts of placenta can be used for derivation of MSC, including, for example, endotheliochorial membrane, chorioallantoic membrane, amniotic membrane, umbilical cord, and Wharton's Jelly.

In embodiments comprising isolation of umbilical cord or placental MSC, the following steps can be performed in a certified clean room, for example under cGMP conditions. In embodiments, tissue is washed extensively in rinsing buffer, then cut into small pieces (1-5 grams). In embodiments, decidual giant cells are removed by one or a combination of steps, for example mechanical scraping of Decidual surface by sterile scoop. The tissue can then be incubated with a protease, for example a serine protease, for example trypsin, for 30-90 minutes at 37ºC and 5% CO₂. Filtration using, for example, nylon mesh, for example 20, 25, and 30 micron nylon mesh, can then be performed. Gradient separation of the cells can then be performed, for example using BSA, Percoll, or Ficoll. In embodiments, differential adhesion is used to allow the quick-attaching cells to be separated from the non-attached cells that are floating in the media. Placenta tissue can then be minced, for example, for 90 sec or 150 cutting cycles.

In disclosed embodiments, tissue, for example placenta tissue, is then subjected to digestion, for example enzymatic digestion, at 37ºC using an enzyme such as collagenase, for 60-180 min while shaking at the rate of, for example, 100 to 140 cycles per minute. Collagenase concentration can range from 1 mg/ml to 5 mg/ml. In embodiments, the cells are then passed through a sequence of cell strainers (for example, 100 micron, 40 micron, etc.) and then through a nylon mesh of, for example, 20, 25 or 30 microns. In embodiments, cells are passed through a gradient. Red Blood Cells (RBC) are removed by RBC lysis buffer (3 min at 4° C.). RBC lysis is neutralized by adding PBS, for example 15-20 times PBS. Cells are then centrifuged at, for example, 400 g for 10 min. Cells are then cultured at a density of, for example, 200-300×10³ per cm² in a culture flask for a culture period of, for example, 5-7 days. In some cases, to remove the remaining giant cells from the mixture, differential adhesion will be applied, and cells will be allowed to attach for a time period such as 1-10 hours, and then the floating cells are separated from the attached cells. After the culture period, placenta MSC can be harvested from the flask as PO (passage zero).

In disclosed embodiments, MSC can be isolated from tissue, for example umbilical cord tissue, in an amount of 1×10⁶ MSC per gram umbilical cord, 2×10⁶ MSC per gram umbilical cord, 3×10⁶ MSC per gram umbilical cord, 4×10⁶ MSC per gram umbilical cord, 5×10⁶ MSC per gram umbilical cord, 6×10⁶ MSC per gram umbilical cord, 7×10⁶ MSC per gram umbilical cord, 8×10⁶ MSC per gram umbilical cord, 9×10⁶ MSC per gram umbilical cord, 1×10⁷ MSC per gram umbilical cord, 2×10⁷ MSC per gram umbilical cord, 3×10⁷ MSC per gram umbilical cord, 4×10⁶ MSC per gram umbilical cord, or the like.

In embodiments, MSC can demonstrate a viability after isolation of greater than 50%, greater than 60%, greater than 70%, greater than 80%, greater than 90%, or the like. In embodiments, MSC can demonstrate a viability after isolation of no less than 50%, no less than 60%, no less than 70%, no less than 80%, no less than 90%, or the like. In embodiments, MSC can demonstrate a viability after isolation of between 50% and 60%, between 60% and 70%, between 70% and 80%, between 80% and 90%, or between 90% and 100%.

MSC can be identified using the minimal criteria established by the Mesenchymal and Tissue Stem Cell Committee of the International Society for Cellular Therapy. These criteria include: first, MSC must be plastic-adherent when maintained in standard culture conditions; second, MSC must express CD105, CD73, and CD90, and lack expression of CD45, CD34, CD14 or CD11b, CD79a or CD19, and HLA-DR surface molecules; and third, MSC must be able to differentiate to osteoblasts, adipocytes and chondroblasts in vitro.

In embodiments, MSC can be isolated based on their ability to produce therapeutic molecules, for example cytokines. For example, Magnetic-Activated Cell Sorting (MACS) can be used to purify MSC based on the cells' ability to produce a particular cytokine. Disclosed embodiments can also comprise the use of flow cytometry to purify MSC based on the cells' ability to produce a particular cytokine. Disclosed embodiments can also comprise the use of chromatography, for example affinity chromatography, to purify MSC based on the cells' ability to produce a particular cytokine.

In embodiments, 100% of the isolated MSC cells express IL-6. In embodiments, expression of IL-6 increases as the cells are passaged. In embodiments, expression of cytokines is up-regulated. For example, in embodiments, expression of IL-6, IL-17A, IFN gamma, TNF alpha, TGF beta, MCP1, HGF, IL-8, TIMP-1, TIMP-2, VEGF, IDO, IL-10, and combinations thereof, can be up-regulated.

In embodiments, isolated MSC are characterized for the expression of surface markers by, for example, flow cytometry, trilineage mesoderm differentiation potential (adipocytes, osteocytes, and chondrocytes), Indoleamine 2,3-dioxygenase (IDO) activity, sterility, endotoxin, and mycoplasma testing.

### Expansion of MSC

In embodiments, cell expansion for cells originating from any of the above-disclosed tissues takes place in clean room facilities purpose built for cell therapy manufacture and meeting GMP clean room classification. In embodiments, cell expansion takes place in a bioreactor, for example a 40 L bioreactor.

For example, in a sterile class II biologic safety cabinet located in a class 10,000 clean production suite, cells are thawed under controlled conditions and washed in a 15 ml conical tube with 10 ML of complete DMEM-low glucose media (cDMEM) (GibcoBRL, Grand Island, N.Y.) supplemented with 10% Fetal Bovine Serum (Hyclone) specified to have endotoxin level less than or equal to 100 EU/mL (with levels routinely less than or equal to 10 EU/mL) and hemoglobin level less than or equal to 30 mg/dl (levels routinely less than or equal to 25 mg/dl). In embodiments, the serum lot used is sequestered and one lot is used for all experiments. In embodiments, the media can be supplemented with, for example, 10% Human Plasmalyte, or Human Serum Albumin, combinations thereof, or the like.

In embodiments, cells are subsequently placed in a T-225 flask containing 25 mL of RB complete medium composed of RoosterNourish-MSC-XF-basal medium and RoosterReplenish-MSC-XF supplement and cultured for 48 hours at 37° C. at 5% CO₂ in a fully humidified atmosphere. Non-adherent cells are washed off using cDMEM by gentle rinsing of the flask. In embodiments, the number of cells plated into the flask can be, for example, between 2.5×10⁵ and 3×10⁶ cells, or between 1.5×10⁶ and 2×10⁶ cells, or the like. In embodiments, adherent cells are subsequently detached by washing the cells with PBS and addition of, for example, 0.05% trypsin containing EDTA (Gibco, Grand Island, N.Y., USA) for 2 minutes at 37° C. at 5% CO₂ in a fully humidified atmosphere. In embodiments, cells can be detached using recombinant compostions, for example TrypLE CTS.

Cells are centrifuged, washed and plated in T-225 flask in 45 mL of cDMEM.

In embodiments, disclosed cell expansion methods can produce between 6 million and 20 million cells per initiating T-225 flask. The cells of the first flask can then be split into, for example, multiple flasks. Cells can then be grown for, for example, 4 days, after which approximately 6 million cells per flask are present (24 million cells total). In embodiments, this method is repeated but cells are not expanded beyond 10 passages, and are then banked in 6 million cell aliquots in sealed vials for delivery.

In further embodiments, cells are grown in media and the cells, along with the media, are recovered after about 2-10 days. The cells are prepared in this “conditioned” media for transfusion at concentrations of less than about 100,000 cells per mL In embodiments, physiological electrolyte additives may be added. In embodiments the cell solution can administered intravenously.

In a further method, cells are grown in media for about 5-10 days. This media is then transfused intravenously without cells or administered locally to the site of an injury. Further methods involve isolation and/or concentration of stem cell produced factors and/or further refinements of these chemicals and/or compounds.

In embodiments, cell proliferation can be expressed in growth per passage. For example, in disclosed embodiments the isolated MSC can increase in number by 40% per passage, 50% per passage, 60% per passage, 70% per passage, 80% per passage, 90% per passage, 100% per passage, 120% per passage, 150% per passage, 200% per passage, 250% per passage, or the like.

In embodiments, cells can be frozen after proliferation, then thawed for further use.

### Activation of MSC

In embodiments, stem cells, for example isolated MSC, can be activated to produce MSC with desired characteristics. For example, MSC can be polarized towards a pro- or anti-inflammatory phenotype depending on the Toll-like receptor (TLR) stimulated. In embodiments, MSC are exposed to stimulatory factors such as inflammatory cytokines. An inflammatory cytokine or proinflammatory cytokine is a type of signaling molecule that is secreted from immune cells like helper T cells (Tₕ) and macrophages, and certain other cell types that promote inflammation. Inflammatory cytokines include interleukin-1 (IL-1), IL-12, IL-17, and IL-18, tumor necrosis factor alpha (TNF-α), interferon gamma (IFNγ), and granulocyte-macrophage colony stimulating factor (GM-CSF). Disclosed embodiments comprise activation of MSC with at least one of IL-1, IL-8, MIP-1b, IL-12, IL-17, IL-18, TNF-α, IFNγ, and GM-CSF. Disclosed embodiments comprise activation of MSC with at least two of IL-1, IL-8, MIP-1b, IL-12, IL-17, IL-18, TNF-α, IFNγ, and GM-CSF. Disclosed embodiments comprise activation of MSC with at least three of IL-1, IL-8, MIP-1b, IL-12, IL-17, IL-18, TNF-α, IFNγ, and GM-CSF. Disclosed embodiments comprise activation of MSC with at least four of IL-1, IL-8, MIP-1b, IL-12, IL-17, IL-18, TNF-α, IFNγ, and GM-CSF.

A disclosed embodiment is described in further detail in the following Examples. In embodiments, the activation amount of each stimulatory factor can be, for example, between 1 ng/ml and 5 ng/ml, or between 2 ng/ml and 4 ng/ml, or the like. In embodiments, the amount of each stimulatory factor can be, for example, 1 ng/ml, 2 ng/ml, 3 ng/ml, 4 ng/ml, 5 ng/ml, 6 ng/ml, 7 ng/ml, 8 ng/ml, 9 ng/ml, 12 ng/ml, 14 ng/ml, 16 ng/ml, 18 ng/ml, 20 ng/ml, 22 ng/ml, 24 ng/ml, 26 ng/mL, 28 ng/ml, 30 ng/ml, 32 ng/ml, 34 ng/ml, 36 ng/ml, 38 ng/ml, 40 ng/ml, 42 ng/ml, 44 ng/ml, 46 ng/mL, or more, or the like. In embodiments, the stimulatory factors are applied in equal amounts. For example, in an embodiment, the stimulatory factors can comprise equal amounts of IL-17, TNF-α, and IFNγ. In an embodiment, the stimulatory factors can comprise different (non-equivalent) amounts of, for example IL-17, TNF-α, and IFNγ.

In embodiments, activation of the MSC comprises contacting the MSC with a stimulatory factor, for example, a cytokine, for example IL-1, IL-8, MIP-1b, IL-12, IL-17, IL-18, TNF-α, IFNγ, or GM-CSF. In embodiments the activation takes place at, for example, 37° C. for a period of time comprising, for example, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more. In embodiments, the activation period can be, for example, between 1 and 20 hours, between 2 and 18 hours, between 3 and 16 hours, between 4 and 14 hours, between 6 and 12 hours, between 8 and 10 hours, or the like. In embodiments, the activation period can be, for example, between 10 and 12 hours. In embodiments, the activation period can be different for different stimulatory factors.

In embodiments, activation of the MSC comprises contacting the MSC with a stimulatory factor, for example, a cytokine, for example IL-1, IL-8, MIP-1b, IL-12, IL-17, IL-18, TNF-α, IFNγ, or GM-CSF. In embodiments the activation takes place at 37° C. for a period of time comprising, for example, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, or the like.

In embodiments, activation of the MSC comprises contacting the MSC with a stimulatory factor, for example, a cytokine, for example IL-1, IL-8, MIP-1b, IL-12, IL-17, IL-18, TNF-α, IFNγ, or GM-CSF. In embodiments the activation takes place at 37° C. for a period of time comprising, for example, not more than 1 hour, a not more than 2 hours, not more than 3 hours, not more than 4 hours, not more than 5 hours, not more than 6 hours, not more than 7 hours, not more than 8 hours, not more than 9 hours, not more than 10 hours, not more than 11 hours, not more than 12 hours, not more than 13 hours, not more than 14 hours, not more than 15 hours, not more than 16 hours, not more than 17 hours, not more than 18 hours, not more than 19 hours, not more than 20 hours, not more than 21 hours, not more than 22 hours, not more than 23 hours, not more than 24 hours, or the like.

In embodiments, activation of the MSC comprises contacting the MSC with a stimulatory factor, for example, a cytokine, for example IL-1, IL-8, MIP-1b, IL-12, IL-17, IL-18, TNF-α, IFNγ, or GM-CSF. In embodiments the activation takes place at RT for a period of time comprising, for example, between 1 and 24 hours, between 2 and 22 hours, between 4 and 18 hours, between 6 and 16 hours, between 8 and 14 hours, between 10 and 12 hours, or the like.

In embodiments, MSC can be frozen after activation, then thawed for further use. In embodiments, MSC can be frozen prior to activation, then thawed and activated.

### Collection of MSC

In embodiments, cell harvesting from the T225 flasks can be performed as follows: the medium, for example Rooster culture, is removed and the flasks are washed with 10 mL D-PBS^{-/-} (Gibco), Th PBS is removed, then 10 mL of CTS-TrypLE (Gibco) is added to the flask, and incubated at 37° C. for 5-6 mins. Media, for example 10 ml of Rooster media, is then added to quench the trypsin activity. In embodiments, the cell suspension is removed and the culture vessel is additionally washed with 25 ml D-PBS^{-/-}. In embodiments, the cell suspension mixture is then centrifuged, for example at 280×g for 10 min at 4° C.

### MSC Compositions

In embodiments, isolated MSC can be formulated into a pharmaceutically-acceptable composition, for example by using at least one pharmaceutically-acceptable carrier. In embodiments, a pharmaceutically-acceptable carrier means a carrier that is useful in preparing a pharmaceutical composition or formulation that is generally safe, non-toxic, and neither biologically nor otherwise undesirable, and includes a carrier that is acceptable for veterinary use as well as human pharmaceutical use. The pharmaceutically acceptable carrier can comprise, for example, saline solution, phosphate buffered saline (PBS), Plasmalyte, Ringer's serum, Ringer's lactate serum, lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oils.

Disclosed formulations comprise MSC combined with cytokines in the form of a composition, e.g., a pharmaceutical composition suitable for administration to a subject in need of treatment with the same.

Disclosed formulations can be “pre-loaded” into administration devices, for example syringes, prior to use.

Disclosed formulations can be provided as a kit. For example, a disclosed kit can comprise a pharmaceutically acceptable carrier; an isolated population of mesenchymal stem cells; isolated interferons, isolated interleukins, and instructions for using the kit in a method for attenuating an immune response. The cell and stimulatory factor, for example, cytokine components of the kit can be administered individually, or combined in vitro and subsequently administered as a mixture. The kit also optionally may include a means of administering the composition, for example by injection.

In embodiments, pelleted hUC-MSCs are resuspended in D-PBS^{-/-} at a concentration of, for example, 1.3×10⁶ cells in 200 uL D-PBS^{-/-}, ensuring that 1×10⁶ hUC-MSCs are injected. In embodiments, the hUC-MSC/D-PBS solution (200 uL) is loaded into one U-100 BD Ultra-Fine Short Insulin Syringes (Beckton, Dickinson, and Company) for a injection in mice, for example a tail vein injection.

### Methods of Treatment Using MSC

Disclosed embodiments can comprise administration of MSC to treat various conditions and diseases. For example, vesicles derived from placental MSC can be employed for therapeutic uses. In embodiments, the stem cells may be autologous to the subject. If available, autologous stem cells can be beneficial to the subject because they reduce or eliminate the potential for adverse immune responses, e.g., rejection of the stem cells or graft-versus-host disease. Autologous stem cells can be, e.g., stem cells isolated directly from the subject (e.g., MSC), or iPS cells produced from non-stem cells from the subject.

In some embodiments, in cases where autologous stem cells are not available or not indicated for a particular subject, allogeneic stem cells can be used. In embodiments, allogeneic stem cells are “matched” as closely as possible to the subject (e.g., via HLA genotype) in order to reduce the likelihood of rejection or graft-versus-host disease. In other embodiments, the stem cell donor is a first-degree-relative (e.g., parent, sibling, or child) of the subject, which increases the likelihood of finding a closely-matched donor. In yet other embodiments, the stem cell donor can be an extended relative of the subject. In some embodiments, the stem cell donor can be from the same race or ethnic group as the subject. However, certain stem cells can be immune-privileged and can be used allogeneically without matching between the donor and subject.

In embodiments, MSC are used for treatment of patients, for example treatment of diseases, conditions, disorders, etc., for example liver disease, and symptoms thereof.

MSC can be administered, for example infused, via any appropriate method, for example subcutaneous, intra-articular, intra-lesional (tendon, ligament, disc), intravenous, intra-peritoneal, or intramuscular administration. In embodiments, administration can comprise, for example, injection. For example, in embodiments, administration can comprise mixing or suspending MSC with, for example blood plasma, HypoThermasol HTS-FRS, Cryostor (containing 0, 2, 5, and 10% DMSO as CSB, CS2, CS5, and CS10, respectively), human serum, human serum albumin, isotonic saline solution 0.7-0.9%, Plasmalyte, Phosphate buffered Solution (PBS), stem cell culture media such as Rooster Replenish CC/RoosterNourish CC, exosome isolation media such as RoosterCollect-EV CC, Infuvite, Lactated Ringer's Solution, and the like. These solutions can be used singularly or in combination with each other.

Appropriate MSC dosage can be, for example, 1×10³ cells, 2.5×10³ cells, 5×10³ cells, 1×10⁴ cells, 2.5×10⁴ cells, 5×10⁴ cells, 1×10⁵ cells, 2.5×10⁵ cells, 5×10⁵ cells, 1×10⁶ cells, 2.5×10⁶ cells, 5×10⁶ cells, 1×10⁷ cells, 2.5×10⁷ cells, 5×10⁷ cells, 1×10⁸ cells, 2.5×10⁸ cells, 5×10⁸ cells, 1×10⁹ cells, 2.5×10⁹ cells, 5×10⁹ cells, 1×10¹⁰ cells, 2.5×10¹⁰ cells, 5×10¹⁰ cells, 1×10¹¹ cells, 2.5×10¹¹ cells, 5×10¹¹ cells, 1×10¹² cells, 2.5×10¹² cells, 5×10¹² cells, 1×10¹³ cells, 2.5×10¹³ cells, 5×10¹³ cells, 1×10¹⁴ cells, 2.5×10¹⁴ cells, 5×10¹⁴ cells, 1×10¹⁵ cells, 2.5×10¹⁵ cells, 5×10¹⁵ cells, or more, or the like.

In embodiments, appropriate MSC dosage can be, for example, between 1×10³ cells and 2.5×10³ cells, between 5×10³ cells and 1×10⁴ cells, between 2.5×10⁴ cells and 5×10⁴ cells, between 1×10⁵ cells and 2.5×10⁵ cells, between 5×10⁵ cells and 1×10⁶ cells, between 2.5×10⁶ cells, between 5×10⁶ cells and 1×10⁷ cells, between 2.5×10⁷ cells and 5×10⁷ cells, between 1×10⁸ cells and 2.5×10⁸ cells, between 5×10⁸ cells and 1×10⁹ cells, between 2.5×10⁹ cells and 5×10⁹ cells, between 1×10¹⁰ cells and 2.5×10¹⁰ cells, between 5×10¹⁰ cells and 1×10¹¹ cells, between 2.5×10¹¹ cells and 5×10¹¹ cells, between 1×10¹² cells and 2.5×10¹² cells, between 5×10¹² cells and 1×10¹³ cells, between 2.5×10¹³ cells and 5×10¹³ cells, between 1×10¹⁴ cells and 2.5×10¹⁴ cells, between 5×10¹⁴ cells and 1×10¹⁵ cells, between 2.5×10¹⁵ cells and 5×10¹⁵ cells, or more, or the like.

In embodiments, appropriate MSC dosage can be, for example, not less than 1×10³ cells, not less than 2.5×10³ cells, not less than 5×10³ cells, not less than 1×10⁴ cells, not less than 2.5×10⁴ cells, not less than 5×10⁴ cells, not less than 1×10⁵ cells, not less than 2.5×10⁵ cells, not less than 5×10⁵ cells, not less than 1×10⁶ cells, not less than 2.5×10⁶ cells, not less than 5×10⁶ cells, not less than 1×10⁷ cells, not less than 2.5×10⁷ cells, not less than 5×10⁷ cells, not less than 1×10⁸ cells, not less than 2.5×10⁸ cells, not less than 5×10⁸ cells, not less than 1×10⁹ cells, not less than 2.5×10⁹ cells, not less than 5×10⁹ cells, not less than 1×10¹⁰ cells, not less than 2.5×10¹⁰ cells, not less than 5×10¹⁰ cells, not less than 1×10¹¹ cells, not less than 2.5×10¹¹ cells, not less than 5×10¹¹ cells, not less than 1×10¹² cells, not less than 2.5×10¹² cells, not less than 5×10¹² cells, not less than 1×10¹³ cells, not less than 2.5×10¹³ cells, not less than 5×10¹³ cells, not less than 1×10¹⁴ cells, not less than 2.5×10¹⁴ cells, not less than 5×10¹⁴ cells, not less than 1×10¹⁵ cells, not less than 2.5×10¹⁵ cells, not less than 5×10¹⁵ cells, or more, or the like.

In embodiments, appropriate MSC dosage can be, for example, not more than 1×10³ cells, not more than 2.5×10³ cells, not more than 5×10³ cells, not more than 1×10⁴ cells, not more than 2.5×10⁴ cells, not more than 5×10⁴ cells, not more than 1×10⁵ cells, not more than 2.5×10⁵ cells, not more than 5×10⁵ cells, not more than 1×10⁶ cells, not more than 2.5×10⁶ cells, not more than 5×10⁶ cells, not more than 1×10⁷ cells, not more than 2.5×10⁷ cells, not more than 5×10⁷ cells, not more than 1×10⁸ cells, not more than 2.5×10⁸ cells, not more than 5×10⁸ cells, not more than 1×10⁹ cells, not more than 2.5×10⁹ cells, not more than 5×10⁹ cells, not more than 1×10¹⁰ cells, not more than 2.5×10¹⁰ cells, not more than 5×10¹⁰ cells, not more than 1×10¹¹ cells, not more than 2.5×10¹¹ cells, not more than 5×10¹¹ cells, not more than 1×10¹² cells, not more than 2.5×10¹² cells, not more than 5×10¹² cells, not more than 1×10¹³ cells, not more than 2.5×10¹³ cells, not more than 5×10¹³ cells, not more than 1×10¹⁴ cells, not more than 2.5×10¹⁴ cells, not more than 5×10¹⁴ cells, not more than 1×10¹⁵ cells, not more than 2.5×10¹⁵ cells, not more than 5×10¹⁵ cells, or more, or the like.

In embodiments, the dose of each MSC injection can be, for example, between 5×10⁶ cells/kg and 5×10⁷ cells/kg.

In embodiments, MSC can be administered one time, two times, three times, four times, five times, every month, or three months, six months or on a yearly basis.

Disclosed methods can also involve the co-administration of bioactive agents with the stem cells. By “co-administration” is meant administration before, concurrently with (e.g., in combination with bioactive agents in the same formulation or in separate formulations), or after administration of a therapeutic composition as described above. As used herein, “bioactive agents” refers to any organic, inorganic, or living agent that is biologically active or relevant. For example, a bioactive agent can be a protein (e.g albumin), a polypeptide, a nucleic acid, a polysaccharide (e.g., heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor-producing virus, a growth factor inhibitor, a growth factor receptor, an anti-inflammatory agent, an antimetabolite, an integrin blocker, or a complete or partial functional sense or antisense gene, including siRNA. It can also include a man-made particle or material, which carries a biologically relevant or active material, for example a nanoparticle comprising a core with a drug and a coating on the core. Bioactive agents can also include drugs such as chemical or biological compounds that can have a therapeutic effect on a biological organism. Non-limiting examples include, but are not limited to, growth factors, anti-rejection agents, anti-inflammatory agents, anti-infective agents (e.g., antibiotics and antiviral agents), and analgesics and analgesic combinations. Anti-inflammatory agents may be useful as additional agents to counteract the inflammatory aspects of the fibrotic process.

Combinations, blends, or other preparations of any of the foregoing examples can be made and still be considered bioactive agents within the intended meaning herein. Aspects of the present disclosure directed toward bioactive agents may include any or all of the foregoing examples. In other embodiments, the bioactive agent may be a growth factor. A growth factor is any agent which promotes the proliferation, differentiation, and functionality of the implanted stem cell. Non-limiting examples of suitable growth factors can include, but are not limited to, leukemia inhibitory factor (LIF), epidermal growth factor (EGF), fibroblast growth factor (FGF), insulin-like growth factor (IGF), vascular endothelial growth factor (VEGF), human growth hormone (hGH), Hepatocyte Growth Factor (HGF), platelet-derived growth factor (PDGF), interleukins, cytokines, and/or combinations thereof. Bioactive agents can be a blood-derived supplement containing mixture of growth factors such as platelet lysate,

In embodiments, the bioactive agent can comprise an immunosuppressive agent. An immunosuppressive agent is any agent which prevents, delays the occurrence of, or decreases the intensity of the undesired immune response, e.g., rejection of a transplanted cell, tissue, or organ, or graft-versus-host disease. Preferred are immunosuppressive agents which suppress cell-mediated immune responses against cells identified by the immune system as non-self. Examples of immunosuppressive agents include, but are not limited to, cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art. In other embodiments, bioactive agents can include anti-fibrotic agents including, but not limited to, nintedanib, INT-767, emricasan, VBY-376, PF-04634817, EXC 001, GM-CT-01, GCS-100, Refanalin, SAR156597, tralokinumab, pomalidomide, STX-100, CC-930, simtuzumab, anti-miR-21, PRM-151, BOT191, palomid 529, IMD1041, serelaxin, PEG-relaxin, ANG-4011, FT011, pirfenidone, F351 (perfenidone derivative), THR-184, CCX-140, FG-3019, avosentan, GKT137831, PF-00489791, pentoxifylline, fresolimumab, and LY2382770.

Disclosed methods of treatment can comprise MSC that have been frozen and thawed, for example, cells can be frozen prior-to or following activation, then thawed for further use.

### EXAMPLES

The following non-limiting examples are provided for illustrative purposes only in order to facilitate a more complete understanding of representative embodiments. These examples should not be construed to limit any of the embodiments described in the present specification.

### Example 1

Fah−/−; Rag2−/−; Il2rgc−/− (FRG) KO liver-humanized mice were generated by crossbreeding of Fah−/− mice (RIKEN) and Rag2−/−; Il2rgc−/−.

The mice were irradiated at 250 kV, 16 mA, 50 cm FSD using a 2 mm filter. The dose exposure rate (cGy) was 150 cGy on a 10 cm by 10 cm field.

Human hematopoietic stem cells (HSC) were collected from fetal liver donor cells, then prepared in sterile media and (one day post-irradiation) injected into the irradiated recipients interhepatically at a dose of 5×10⁵ cells per mouse.

Animals were fed with autoclaved/irradiated food and acidified autoclaved water with or without SMZ (7.8 ml of SMZ per 250 ml of drinking water) on alternate weeks for the duration of their lives after weaning.

Blood was collected from the facial vein to detect the human stem cell xenografted mice.

100 μl of blood per mouse was collected in 1.5-ml sterile microcentrifuge tubes containing 100 μl of 20 mM PBS-EDTA and placed on ice. PBMC's were resuspended in red blood cell lysis buffer (1×ACK lysis buffer), and incubated for 5 min at RT (25° C.).

Cells were centrifuged at 469×g twice and resuspended in 2% (vol/vol) FBS/PBS containing human CD45, mouse CD45 antibodies, and 7-AAD mixture. The human immune reconstitution (% of human CD45+ cells/total CD45+ cells) was examined using flow cytometry analyses.

Human Umbilical cord mesenchymal stromal cells (hUC-MSCs) were isolated from the perivascular Wharton's Jelly region of the human umbilical cord, and characterized for the expression of surface markers by flow cytometry, trilineage mesoderm differentiation potential (adipocytes, osteocytes, and chondrocytes), Indoleamine 2,3-dioxygenase (IDO) activity, sterility, endotoxin, and mycoplasma test. The hUC-MSCs were cultured and harvested following RB manufacturing protocols. One million five hundred thousand to two million hUC-MSCs were plated in T225 vented flasks in 25 ml of RB complete medium and cultured for 48 hours at 37° ° C. with 5% CO₂.

After initial culture of hUC-MSCs, for example between 20 and 50 hours, or between 36-38 hours after initial culture of hUC-MSCs, activation consisting of human TNF-α, human IFNγ, and human IL-17 was added to each T225 Flask with hUC-MSCs at a final concentration of 2 ng/ml for each cytokine. The flasks can be cultured with added activation media for an additional, for example, between 2 and 20 hours, such as between 10 and 12 hours at 37° C. with 5% CO₂.

Cells were then harvested. Rooster culture medium was removed and the cells washed with 10 mL D-PBS^{-/-} then media was removed. 10 mL of CTS-TrypLE was added to the flasks and incubated for 5-6 mins at 37° C. 10 ml of media was then added to quench the trypsin activity. The cell suspension was removed and the flasks additionally washed with 25 ml D-PBS^{-/-}. The cell suspension mixture was centrifuged at 280×g for 10 min at 4° C.

Pelleted hUC-MSCs were resuspended in D-PBS^{-/-} at 1,300,000 cells in 200 uL D-PBS^{-/-} to ensure that 1,000,000 hUC-MSCs are injected. The hUC-MSC/D-PBS solution (200 uL) was loaded into one U-100 BD Ultra-Fine Short Insulin Syringes for a tail vein injection. Each mice was injected with 5 doses at 1,000,000 cells/injection in 3 weeks; during the first week they were injected once on day 0, day 2, and day 5, then injected once weekly for additional 2 weeks.

### Cohort 1—

50 mice were fed modified high-fat Lieber-DeCarli (L-D) liquid diet with alcohol (3.5% w/v) and high-fat or isocaloric dextrin for 4 weeks (75 to 103 days of age). During feeding of HFCD, mice were given a 53% ethanol solution in water twice a week by oral gavage at a dosage 4 g/kg ethanol as for a total of 8 times. For the maltose control group, the 1-month HFCD-fed mice were simultaneously given isocaloric dextrin-maltose by oral gavage twice a week for a total of 8 times. 31 mice died prior to treatment and following attempts to obtain imaging with CT+/− ultrasound elastography. After preparation with alcohol and an attempt to obtain Computerized Tomography Imaging with sedation, 19 humanized mice at age 104 days remained to randomize and complete the cohort 1 studies (10 males and 9 females).

Mice were randomly assigned to placebo (PBS) (n=5) or non-activated MSC (n=14) and PBS or MSC administered according to the protocol. Of the 14 treated mice, 8 received the MSC IV and IP and 6 received IP only.

Group 1: 5 mice were administered vehicle (PBS) IP and IV;

Group 2: 8 mice were administered 1,000,000 non-activated mesenchymal stem cell therapy Intravenously (IV) and intraperitoneally (IP);

Group 3: 6 mice were administered 1,000,000 non-activated mesenchymal stem cell therapy intraperitoneally (IP).

Alcohol binge was administered bi-weekly for three weeks. MSCs or PBS was administered to mice three times on the first week and two times each week for the next two weeks (eight times during three-week).

### Cohort 2—

After analyzing the initial data, binge drinking timing was shortened, and no attempt was made to obtain Computerized Tomography scans. An additional 33 humanized mice that underwent binge alcohol preparation for three weeks were divided into 5 groups:

Group 1: 5 mice received 1,000,000 non-activated mesenchymal cells IV and IP;

Group 2: 7 mice received IV and IP vehicle (PBS);

Group 3: 7 mice received 1,000,000 activated mesenchymal cells IP;

Group 4: 7 mice received 1,000,000 activated mesenchymal cells IV;

Group 5: 7 mice received 1,000,000 activated mesenchymal cells IV and IP.

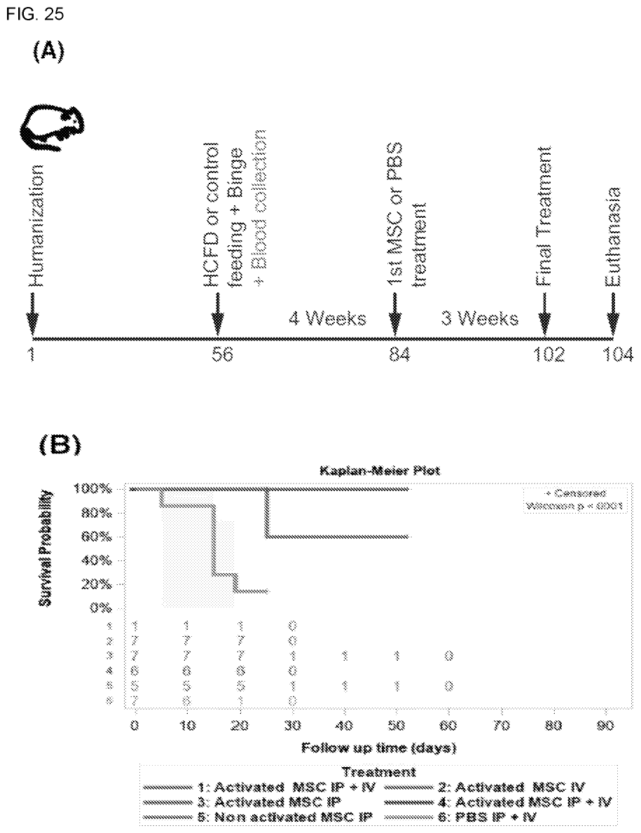

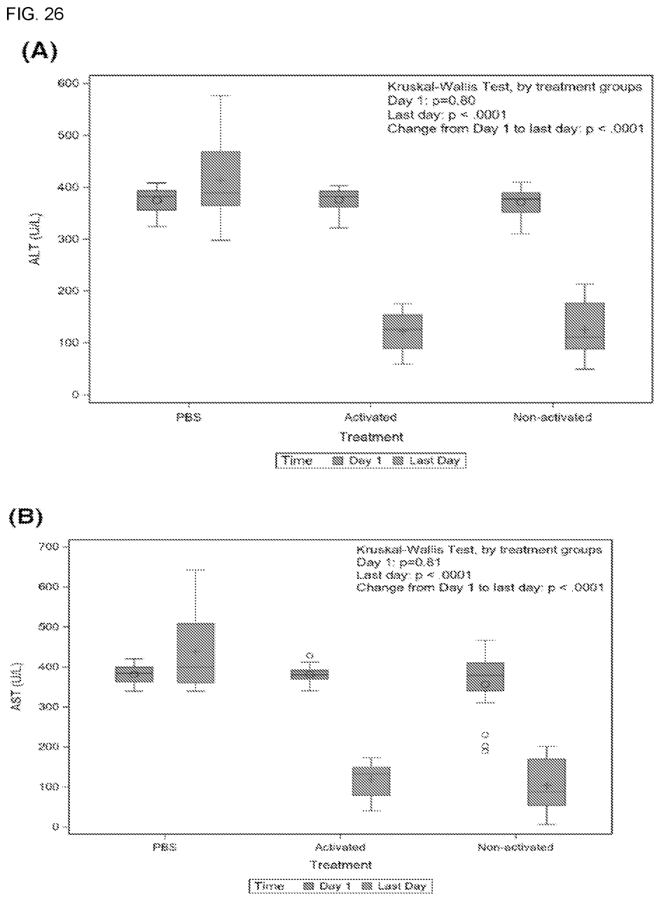

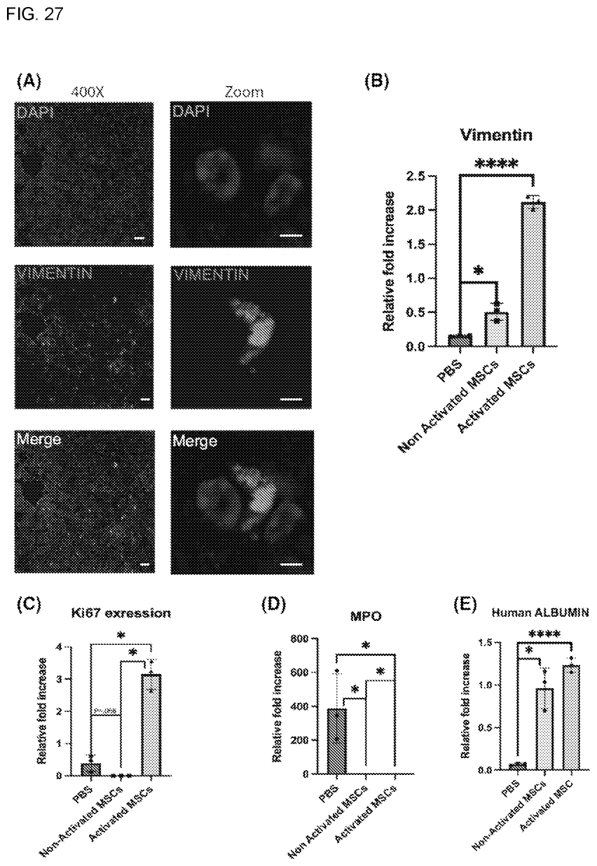

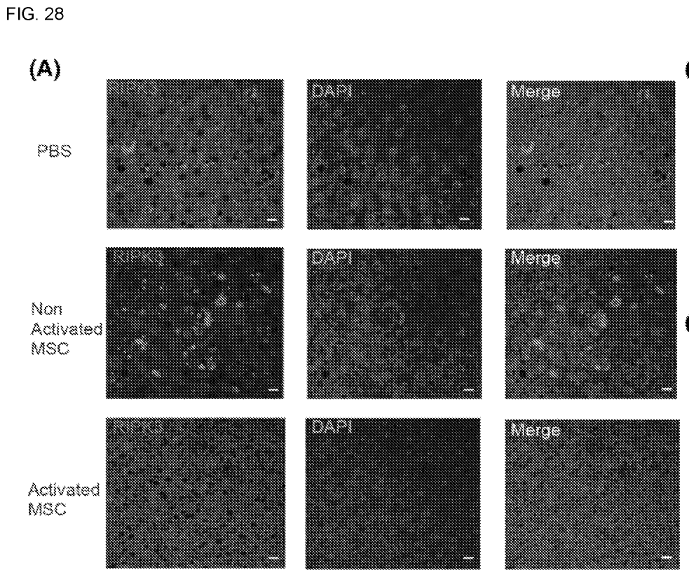

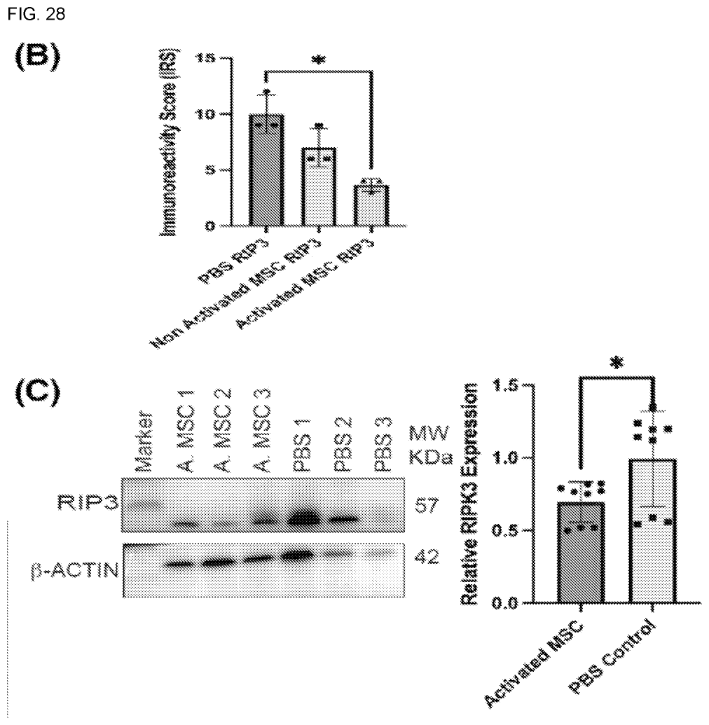

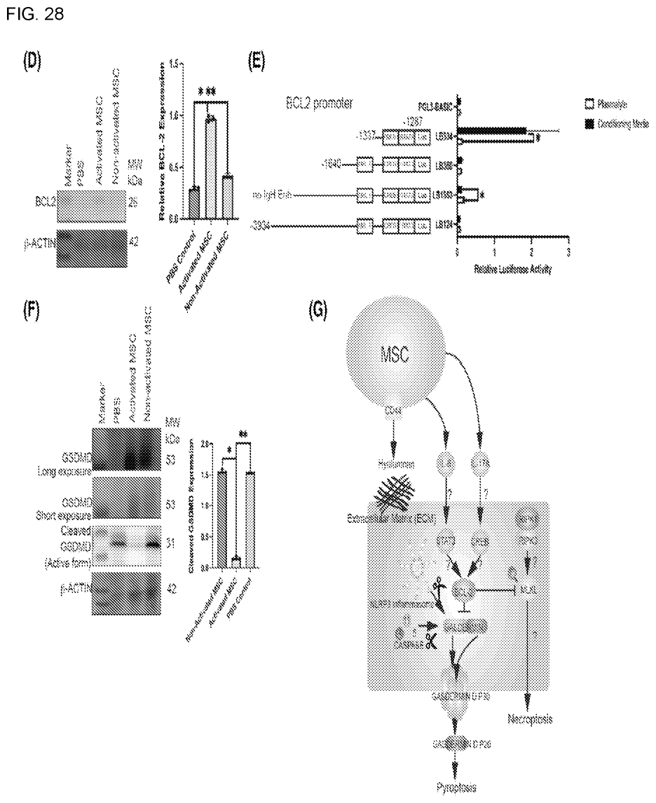

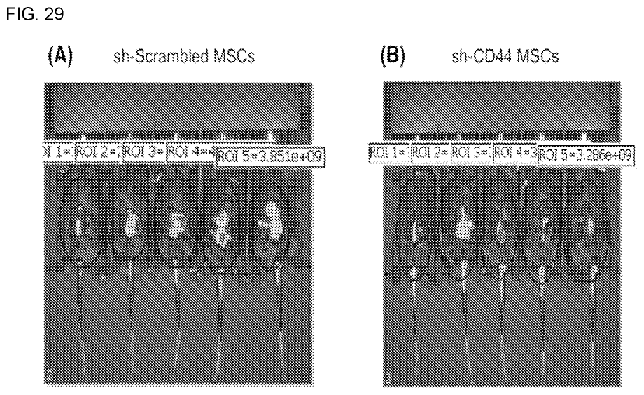

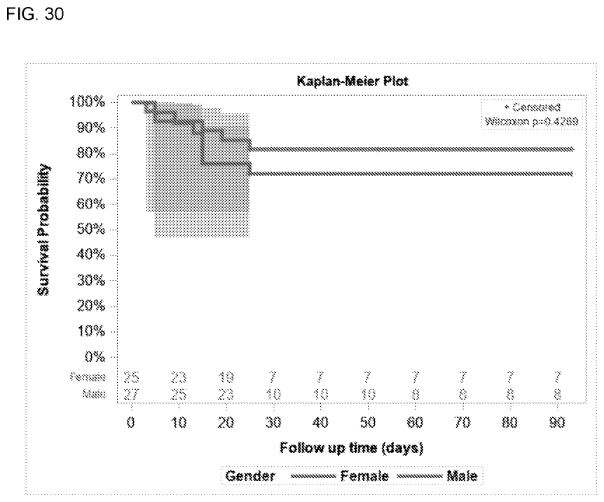

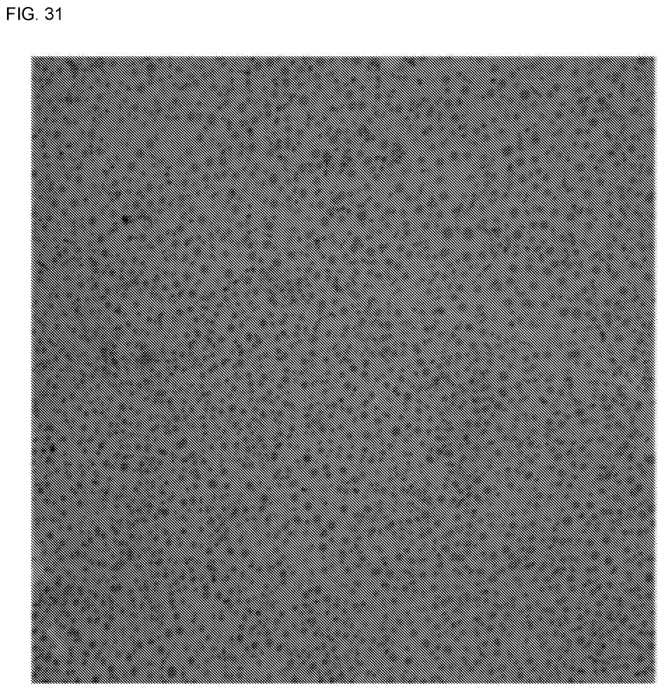

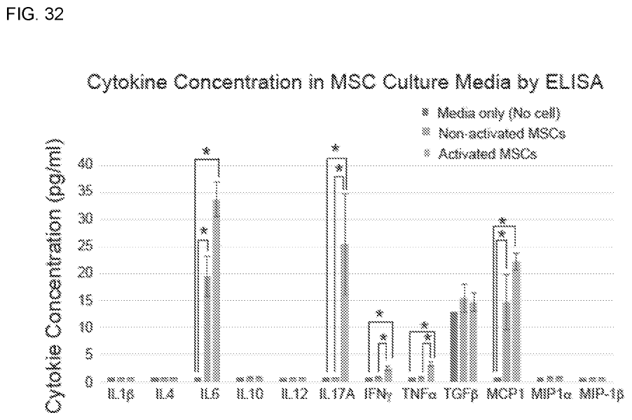

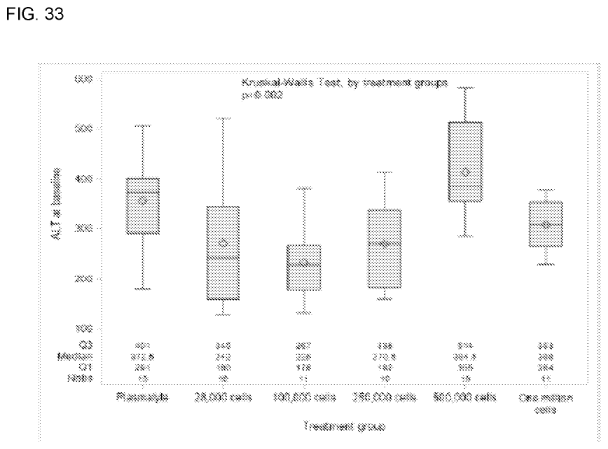

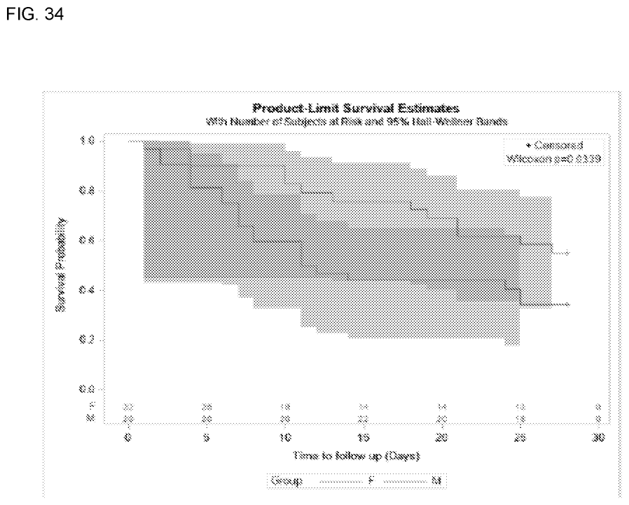

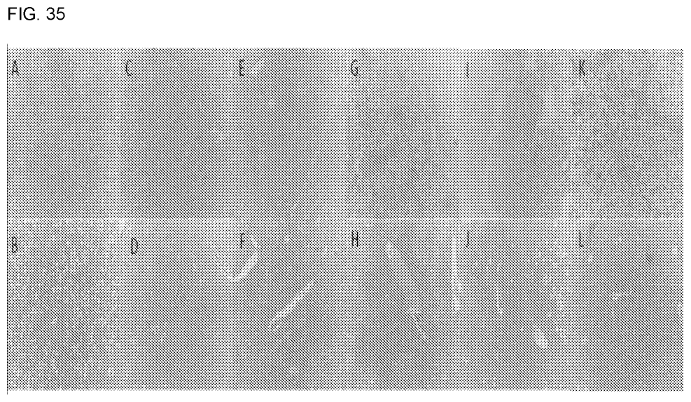

Cells/PBS were administered three times during the first week and once a week for an additional two weeks (FIG. 25A).

For both cohorts, blood samples were obtained on the first day of treatment and on the day of death or euthanasia. The samples were sent for measurement of ALT and AST levels.

For cohort 1, mice were observed for survival up to 93 days after initiation of MSC or PBS treatment. Surviving mice were euthanized at the end of this experiment by cardiopuncture and cervical dislocation. Liver tissues were fixed with neutral buffered 10% formalin for H&E staining and histological evaluation of the tumor.

For cohort 2, mice were observed for survival up to 25 days after initiation of MSC or PBS treatment. Surviving mice were euthanized in the same manner 2 days after final MSC treatment. Necropsy was performed on mice that died before endpoint.

Blood collected from FRG-hu HSC/Hep mice was used to quantify human leukocyte reconstitution at 4 weeks post infection and at euthanasia endpoint, 60 days after initiation of MSC treatment (day 167). The blood was collected to measure human transplant efficacy of fetal liver cells.

After euthanasia, a representative section of liver tissue was fixed with neutral buffered 10% formalin and processed for histological evaluation. The degree of steatosis, necrosis, as well as fibrosis was quantified by blinded specimen analysis by representative hematoxylin eosin-stained section examination.

Steatosis was graded on a 4-tier score (0-3) with 0 being <5% steatosis, 1 being 5-33%, 2 being 34-66% and 3 being >66%. Necrosis was also graded on a 4-tier score (0-3) with 0 being <5% necrosis, 1 being 5-10%, 2 being < or equal to 20% and 3 being >21%.

Log-rank (Mantel-Cox) test, Gehan-Breslow-Wilcoxon test and Chi square test were used to calculate statistical values for mouse survival studies. For mouse histological studies, ANOVA test or Student's T test was used.

Table 1 includes the details of dosing, survival, pathology and AST and ALT levels for all 52 mice that were randomized and completed the study.

TABLE 1
Table 1. Individual humanized FRG mice preclinical trial datasheet with
liver enzyme levels in serum in the presence or absence of MSC treatment

| | | | | | | | | ALT at | | AST at |
|---|---|---|---|---|---|---|---|---|---|---|
| | | | | | Survival | | ALT | Death/ | AST | Death/ |
| Co- | | Treatment/ | Day last | Day of | to Eutha- | | Day 1 | Eutha- | Day 1 | Eutha- |
| hort | Mouse | Route | Treatment | Death | nasia | Pathology | Treatment | nasia | Treatment | nasia |
| 1 | 1 | Non activated MSC | Day 24 | Day 93 | Yes | NSF | 310 | 213 | 230 | 197.1 |
| | | IV + IP | | | | | | | | |
| 1 | 2 | Non activated MSC | Day 24 | Day 93 | Yes | NSF | 350 | 49 | 189 | 27.14 |
| | | IV + IP | | | | | | | | |
| 1 | 3 | Non activated MSC | Day 24 | Day 93 | Yes | NSF | 345 | 95 | 202 | 53.7 |
| | | IV + IP | | | | | | | | |
| 1 | 4 | Non activated MSC | Day 24 | Day 93 | Yes | NSF | 399 | 143 | 310 | 86 |
| | | IV + IP | | | | | | | | |
| 1 | 5 | Non activated MSC | Day 24 | Day 93 | Yes | NSF | 402 | 88 | 410 | 53.7 |
| | | IV + IP | | | | | | | | |
| 1 | 6 | Non activated MSC | Day 24 | Day 93 | Yes | NSF | 389 | 74 | 379 | 19.7 |
| | | IV + IP | | | | | | | | |
| 1 | 7 | Non activated MSC | Day 24 | Day 93 | Yes | NSF | 387 | 86 | 410 | 6 |
| | | IV + IP | | | | | | | | |
| 1 | 8 | Non activated MSC | Day 24 | Day 93 | Yes | NSF | 369 | 98 | 375 | 84 |
| | | IV + IP | | | | | | | | |
| 1 | 9 | Non activated MSC | Day 24 | Day 93 | Yes | NSF | 345 | 65 | 350 | 19.7 |
| | | IV + IP | | | | | | | | |
| 1 | 10 | Non activated MSC | Day 24 | Day 93 | Yes | absent to mild mixed portal | 409 | 175 | 410 | 120 |
| | | IV + IP | | | | and perivenular inflammation | | | | |
| 1 | 11 | Non activated MSC | Day 24 | Day 93 | Yes | absent to mild perivenular | 38 [symbol] | 184 | 465 | 115 |
| | | IV + IP | | | | inflammation | | | | |
| 1 | 12 | Non activated MSC | Day 24 | Day 93 | Yes | rare focus of lobular | 377 | 110 | 389 | 97 |
| | | IV + IP | | | | inflammation | | | | |
| 1 | 13 | Non activated MSC | Day 24 | Day 93 | Yes | NSF | 381 | 90 | 367 | 79 |
| | | IV + IP | | | | | | | | |
| 1 | 14 | Non activated MSC | Day 24 | Day 93 | Yes | NSF | 357 | 102 | 389 | 88 |
| | | IV + IP | | | | | | | | |
| 1 | 15 | PBS IP | Day 3 | Day 3 | Yes | 2 + steatosis/mild lobular | 392 | 475 | 401 | 642 |
| | | | | | | inflammation | | | | |
| 1 | 16 | PBS IP | Day 9 | Day 9 | No | 2 + steatosis, mild lobular | | | | |
| | | | | | | inflammation, Perivenular | | | | |
| | | | | | | necrosis −5-10% of the | | | | |
| | | | | | | submitted tissue | 349 | 542 | 389 | 583 |
| 1 | 17 | PBS IP | Day 24 | Day 93 | Yes | minimal steatosis, 5% | 408 | 389 | 377 | 45 [symbol] |
| 1 | 18 | PBS IP | Day 5 | Day 5 | No | <5% steatosis | 398 | 676 | 394 | [symbol] 4 |
| 1 | 19 | PBS IP | Day 13 | Day 13 | No | 2 + steatosis, mild lobular | 395 | 481 | 37 [symbol] | 453 |
| | | | | | | inflammation | | | | |
| 2 | 20 | PBS IP + IV | Day 18 | Day 19 | No | <5% steatosis | 387 | 298 | 398 | 381 |
| 2 | 21 | PBS IP + IV | Day 23 | Day 25 | Yes | NSF | 387 | 315 | 407 | 347 |
| 2 | 22 | PBS IP + IV | Day 11 | Day 15 | No | 1 + steatosis | 324 | 358 | 420 | 342 |
| 2 | 23 | PBS IP + IV | Day 11 | Day 15 | No | NSF | 389 | 387 | 379 | 339 |
| 2 | 24 | PBS IP + IV | Day 4 | Day 5 | No | 1 + steatosis, 5-10% necrosis | 349 | 392 | 34 [symbol] | 404 |
| 2 | 25 | PBS IP + IV | Day 11 | Day 15 | No | NSF | 378 | 372 | 348 | 374 |
| 2 | 26 | PBS IP + IV | Day 11 | Day 15 | No | No steatosis, perivenular | 382 | 389 | 33 [symbol] | 395 |
| | | | | | | necrosis, approximately 5-10% | | | | |
| | | | | | | necrosis | | | | |
| 2 | 27 | Activated MSC | Day 23 | Day 25 | Yes | 1 + steatosis | 389 | 89 | 364 | 73 |
| | | IP + IV | | | | | | | | |
| 2 | 28 | Activated MSC | Day 23 | Day 25 | Yes | 1 + steatosis | 367 | 99 | 391 | [symbol] |
| | | IP + IV | | | | | | | | |
| 2 | 29 | Activated MSC | Day 23 | Day 25 | Yes | NSF | 384 | 78 | 370 | 49 |
| | | IP + IV | | | | | | | | |
| 2 | 30 | Activated MSC | Day 23 | Day 25 | Yes | 1 + steatosis | 392 | 88 | 370 | 74 |
| | | IP + IV | | | | | | | | |
| 2 | 31 | Activated MSC | Day 23 | Day 25 | Yes | <5% steatosis | 403 | 6 [symbol] | 382 | 40 |
| | | IP + IV | | | | | | | | |
| 2 | 32 | Activated MSC | Day 23 | Day 25 | Yes | NSF | 39 [symbol] | 5 [symbol] | 412 | 78 |
| | | IP + IV | | | | | | | | |
| 2 | 33 | Activated MSC | Day 23 | Day 25 | Yes | 1 + steatosis | 382 | 67 | 384 | 102 |
| | | IP + IV | | | | | | | | |
| 2 | 34 | Activated MSC IP | Day 23 | Day 25 | Yes | NSF | 392 | 154 | 374 | 184 |
| 2 | 35 | Activated MSC IP | Day 23 | Day 25 | Yes | 1 + steatosis | 354 | 126 | 358 | 137 |
| 2 | 36 | Activated MSC IP | Day 23 | Day 25 | Yes | <5% steatosis | 382 | 167 | 380 | 173 |
| 2 | 37 | Activated MSC IP | Day 23 | Day 25 | Yes | <5% steatosis | 321 | 128 | 405 | 143 |
| 2 | 38 | Activated MSC IP | Day 23 | Day 25 | Yes | 1 + steatosis | 369 | 15 [symbol] | 378 | 130 |
| 2 | 39 | Activated MSC IP | Day 23 | Day 25 | Yes | <5% steatosis: | 382 | 11 [symbol] | 3 [symbol] 9 | 120 |
| 2 | 40 | Activated MSC IP | Day 23 | Day 25 | Yes | 1 + steatosis | 348 | 1 [symbol] 6 | 340 | 152 |
| 2 | 41 | Activated MSC IV | Day 23 | Day 25 | Yes | 2 + steatosis | 392 | 143 | 340 | 132 |
| 2 | 42 | Activated MSC IV | Day 23 | Day 25 | Yes | 1 + steatosis | 378 | 13 [symbol] | 397 | 142 |
| 2 | 43 | Activated MSC IV | Day 23 | Day 25 | Yes | <5% steatosis | 401 | 153 | 390 | 119 |
| 2 | 44 | Activated MSC IV | Day 23 | Day 25 | Yes | <5% steatosis | 39 [symbol] | 115 | 410 | 148 |
| 2 | 45 | Activated MSC IV | Day 23 | Day 25 | Yes | 1 + steatosis, 10-20% necrosis, | 38#z,899 | 163 | 428 | 1 [symbol] 3 |
| | | | | | | marked portal inflammation | | | | |
| | | | | | | and few balloon cells | | | | |
| 2 | 46 | Activated MSC IV | Day 23 | Day 25 | Yes | NSF | 3 [symbol] 2 | 110 | 385 | 149 |
| 2 | 47 | Activated MSC IV | Day 23 | Day 25 | Yes | NSF | 341 | 175 | 374 | 153 |
| 2 | 48 | Non activated MSC IP | Day 23 | Day 25 | Yes | NSF | 370 | 164 | 396 | 187 |
| 2 | 49 | Non activated MSC IP | Day 23 | Day 25 | Yes | NSF | 352 | 136 | 340 | 170 |
| 2 | 50 | Non activated MSC IP | Day 1 | Day 5 | No | 1 + steatosis, 30% necrosis | 354 | 149 | 411 | 189 |
| 2 | 51 | Non activated MSC IP | Day 1 | Day 5 | No | <5% steatosis | 382 | 17 [symbol] | 380 | 201 |
| 2 | 52 | Non activated MSC IP | Day 23 | Day 25 | Yes | NSF | 384 | 186 | 369 | 187 |

[symbol]

indicates data missing or illegible when filed

Table 2 provides details of the mice pertaining to sex, treatment, survival, AST and ALT levels, and histology:

TABLE 1
Individual humanized FRG mice preclinical trial datasheet with liver enzyme levels in serum in the presence or absence of MSC treatment

| | | | | | | | | ALT at | | AST at |
|---|---|---|---|---|---|---|---|---|---|---|
| | | | | | Survival | | ALT | Death/ | AST | Death/ |
| Co- | | Treatment/ | Day last | Day of | to Eutha- | | Day 1 | Eutha- | Day 1 | Eutha- |
| hort | Mouse | Route | Treatment | Death | nasia | Pathology | Treatment | nasia | Treatment | nasia |
| 1 | 1 | Non activated MSC | Day 24 | Day 93 | Yes | NSF | 310 | 213 | 230 | 197.1 |
| | | IV + IP | | | | | | | | |
| 1 | 2 | Non activated MSC | Day 24 | Day 93 | Yes | NSF | 350 | 49 | 189 | 27.14 |
| | | IV + IP | | | | | | | | |
| 1 | 3 | Non activated MSC | Day 24 | Day 93 | Yes | NSF | 345 | 95 | 202 | 53.7 |
| | | IV + IP | | | | | | | | |
| 1 | 4 | Non activated MSC | Day 24 | Day 93 | Yes | NSF | 3 [symbol] | 143 | 310 | 8 [symbol] |
| | | IV + IP | | | | | | | | |
| 1 | 5 | Non activated MSC | Day 24 | Day 93 | Yes | NSF | 402 | 88 | 410 | 53.7 |
| | | IV + IP | | | | | | | | |
| 1 | 6 | Non activated MSC | Day 24 | Day 93 | Yes | NSF | 389 | 74 | 379 | 19.7 |
| | | IV + IP | | | | | | | | |
| 1 | 7 | Non activated MSC | Day 24 | Day 93 | Yes | NSF | 387 | 86 | 410 | 6 |
| | | IV + IP | | | | | | | | |
| 1 | 8 | Non activated MSC | Day 24 | Day 93 | Yes | NSF | 369 | 98 | 375 | 84 |
| | | IV + IP | | | | | | | | |
| 1 | 9 | Non activated MSC | Day 24 | Day 93 | Yes | NSF | 345 | 65 | 3 [symbol] 0 | 19.7 |
| | | IV + IP | | | | | | | | |
| 1 | 10 | Non activated MSC | Day 24 | Day 93 | Yes | absent to mild mixed portal | 409 | 175 | 410 | 120 |
| | | IV + IP | | | | and perivenular inflammation | | | | |
| 1 | 11 | Non activated MSC | Day 24 | Day 93 | Yes | absent to mild perivenular | 388 | 184 | 486 | 115 |
| | | IV + IP | | | | inflammation | | | | |
| 1 | 12 | Non activated MSC | Day 24 | Day 93 | Yes | rare focus of lobular | 377 | 110 | 38 [symbol] | [symbol] 7 |
| | | IV + IP | | | | inflammation | | | | |
| 1 | 13 | Non activated MSC | Day 24 | Day 93 | Yes | NSF | 391 | 90 | 367 | 7 [symbol] |
| | | IV + IP | | | | | | | | |
| 1 | 14 | Non activated MSC | Day 24 | Day 93 | Yes | NSF | 357 | 102 | 389 | 88 |
| | | IV + IP | | | | | | | | |
| 1 | 15 | PBS IP | Day 3 | Day 3 | Yes | 2 + steatosis/mild lobular | 392 | 475 | 401 | 642 |
| | | | | | | inflammation | | | | |
| 1 | 16 | PBS IP | Day 9 | Day 9 | No | 2 + steatosis, mild lobular | | | | |
| | | | | | | inflammation, Perivenular | | | | |
| | | | | | | necrosis −5-10% of the | | | | |
| | | | | | | submitted tissue | 34 [symbol] | 542 | 38 [symbol] | 583 |
| 1 | 17 | PBS IP | Day 24 | Day 93 | Yes | minimal steatosis, 5% | 408 | 389 | 377 | 45 [symbol] |
| 1 | 18 | PBS IP | Day 5 | Day 5 | No | <5% steatosis | 398 | [symbol] 76 | 394 | 5 [symbol] 4 |
| 1 | 19 | PBS IP | Day 13 | Day 13 | No | 2 + steatosis, mild lobular | 395 | 4 [symbol] 1 | 378 | 453 |
| | | | | | | inflammation | | | | |
| 2 | 20 | PBS IP + IV | Day 18 | Day 19 | No | <5% steatosis | 387 | 298 | 398 | 381 |
| 2 | 21 | PBS IP + IV | Day 23 | Day 25 | Yes | NSF | 387 | 315 | 407 | 347 |
| 2 | 22 | PBS IP + IV | Day 11 | Day 15 | No | 1 + steatosis | 324 | 358 | 420 | 342 |
| 2 | 23 | PBS IP + IV | Day 11 | Day 15 | No | NSF | 389 | 387 | 379 | 339 |
| 2 | 24 | PBS IP + IV | Day 4 | Day 5 | No | 1 + steatosis 5-10% necrosis | 349 | 392 | 348 | 404 |
| 2 | 25 | PBS IP + IV | Day 11 | Day 15 | No | NSF | 378 | 372 | 348 | 374 |
| 2 | 26 | PBS IP + IV | Day 11 | Day 15 | No | No steatosis, perivenular | 3 [symbol] 2 | 389 | 339 | 395 |
| | | | | | | necrosis, approximately 5-10% | | | | |
| | | | | | | necrosis | | | | |
| 2 | 27 | Activated MSC | Day 23 | Day 25 | Yes | 1 + steatosis | 389 | 89 | 364 | 73 |
| | | IP + IV | | | | | | | | |
| 2 | 28 | Activated MSC | Day 23 | Day 25 | Yes | 1 + steatosis | 367 | 99 | 391 | 8 [symbol] |
| | | IP + IV | | | | | | | | |
| 2 | 29 | Activated MSC | Day 23 | Day 25 | Yes | NSF | 384 | 78 | 370 | 49 |
| | | IP + IV | | | | | | | | |
| 2 | 30 | Activated MSC | Day 23 | Day 25 | Yes | 1 + steatosis | 392 | 88 | 370 | 74 |
| | | IP + IV | | | | | | | | |
| 2 | 31 | Activated MSC | Day 23 | Day 25 | Yes | <5% steatosis | 403 | [symbol] | 382 | 40 |
| | | IP + IV | | | | | | | | |
| 2 | 32 | Activated MSC | Day 23 | Day 25 | Yes | NSF | 396 | 5 [symbol] | 412 | 78 |
| | | IP + IV | | | | | | | | |
| 2 | 33 | Activated MSC | Day 23 | Day 25 | Yes | 1 + steatosis | 382 | 87 | 384 | 102 |
| | | IP + IV | | | | | | | | |
| 2 | 34 | Activated MSC IP | Day 23 | Day 25 | Yes | NSF | 392 | 154 | 374 | 184 |
| 2 | 35 | Activated MSC IP | Day 23 | Day 25 | Yes | 1 + steatosis | 354 | 12 [symbol] | 356 | 137 |
| 2 | 36 | Activated MSC IP | Day 23 | Day 25 | Yes | <5% steatosis | 382 | 167 | 380 | 173 |
| 2 | 37 | Activated MSC IP | Day 23 | Day 25 | Yes | <5% steatosis | 321 | 128 | 405 | 143 |
| 2 | 38 | Activated MSC IP | Day 23 | Day 25 | Yes | 1 + steatosis | 369 | 159 | 378 | 130 |
| 2 | 39 | Activated MSC IP | Day 23 | Day 25 | Yes | <5% steatosis: | 382 | 119 | 3 [symbol] 9 | 120 |
| 2 | 40 | Activated MSC IP | Day 23 | Day 25 | Yes | 1 + steatosis | 348 | 186 | 340 | 152 |
| 2 | 41 | Activated MSC IV | Day 23 | Day 25 | Yes | 2 + steatosis | 392 | 143 | 340 | 132 |
| 2 | 42 | Activated MSC IV | Day 23 | Day 25 | Yes | 1 + steatosis | 378 | 13 [symbol] | 397 | 142 |
| 2 | 43 | Activated MSC IV | Day 23 | Day 25 | Yes | <5% steatosis | 401 | 153 | 390 | 119 |
| 2 | 44 | Activated MSC IV | Day 23 | Day 25 | Yes | <5% steatosis | 3 [symbol] | 115 | 410 | 148 |
| 2 | 45 | Activated MSC IV | Day 23 | Day 25 | Yes | 1 + steatosis, 10-20% necrosis, | 386 | 163 | 428 | 163 |
| | | | | | | marked portal inflammation | | | | |
| | | | | | | and few balloon cells | | | | |
| 2 | 46 | Activated MSC IV | Day 23 | Day 25 | Yes | NSF | 3 [symbol] 2 | 110 | 385 | 149 |
| 2 | 47 | Activated MSC IV | Day 23 | Day 25 | Yes | NSF | 341 | 175 | 374 | 153 |
| 2 | 48 | Non activated MSC IP | Day 23 | Day 25 | Yes | NSF | 370 | 184 | 396 | 187 |
| 2 | 49 | Non activated MSC IP | Day 23 | Day 25 | Yes | NSF | 352 | 136 | 340 | 170 |
| 2 | 50 | Non activated MSC IP | Day 1 | Day 5 | No | 1 + steatosis, 30% necrosis | 354 | 149 | 411 | 189 |
| 2 | 51 | Non activated MSC IP | Day 1 | Day 5 | No | <5% steatosis | 382 | 176 | 380 | 201 |
| 2 | 52 | Non activated MSC IP | Day 23 | Day 25 | Yes | NSF | 384 | 186 | 3 [symbol] 9 | 187 |

Note:
Table showing Cohort 1 (N = 19 mice) and 2 (N = 52 mice) preclinical data. [symbol]
indicates data missing or illegible when filed

Table 3 shows the primer sets used for qPCR. Primer sets were ordered from Integrated DNA technologies (IDT):

| Gene | | |
|---|---|---|
| Name | | Sequence |
| Human | Sense | 5′-GAA AGC CCA CTG TCT TAG TG-3 |
| Albumin | Anti- | 5′-GGG TGT AGC GAA CTA GAA TG-3 |
| | Sense | |
| Mouse | Sense | 5-GCA AGG CTG CTG ACA AGG A-3 |
| Albumin | Anti- | 5-GGC GTC TTT GCA TCT AGT GAC-A-3 |
| | Sense | |
| Ki-67 | Sense | 5- TCC TTT GGT GGG CAC CTA AGA CCT |
| | | G-3 |
| | Anti- | 5- TGA TGG TTG AGG TCG TTC CTT GAT |
| | Sense | G-3 |
| MPO | Sense | 5- CCA ACA ACA TCG ACA TCT GG-3 |
| | Anti- | 5- GCT GAA CAC ACC CTC GTT CT-3 |
| | Sense | |
| Vimentin | Sense | 5- CCA CCA GGT CCG TGT CCT CGT-3 |
| | Anti- | 5- CGC TGC CCA GGC TGT AGG TG-3 |
| | Sense | |

### Cohort 1—

Four (of 5) control mice died on days 3, 5, 9 and 13 post-randomization and first treatment. None of the 14 mice treated with non-activated MSC died during the experiment. All surviving animals were sacrificed 93 days after randomization.

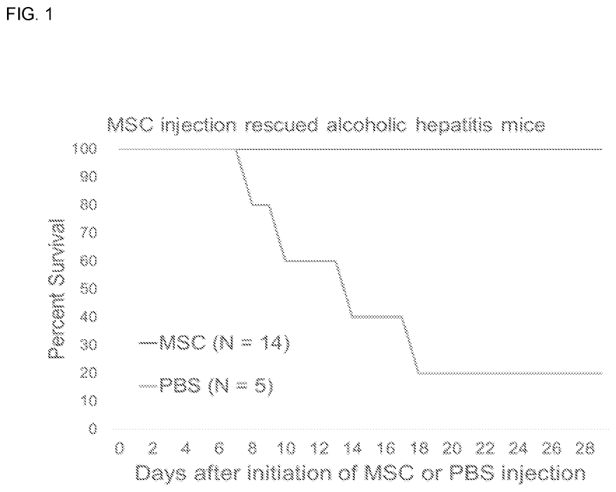

After four weeks of Mesenchymal Stem cell therapy (PrimeGen) MSC injection group (n=14) had a high survival rate compared to PBS control group (n=5) (FIG. 1). Mantel-Cox and Gehan-Breslow-Wilcoxen test showed statistical significance with p<0.0001.

Pathology was mixed and with varying degrees of steatosis and only 6 animals demonstrating anywhere between 5 and 10% necrosis. No fibrosis was appreciated on HE stained sections. On Table 1, all control mice (n=5) had some degree of steatosis and 3 had some degree of lobular inflammation. Of the 14 mice treated with MSC, 11 had no steatosis or any other significant findings and 3 had no steatosis and only minimal inflammation. Of note, the treated mice, all of which survived, were euthanized over 2 months after the last injection which may have been too late to see the damage as the surviving mouse would have healed at that point. This was considered in the next set of experiments.

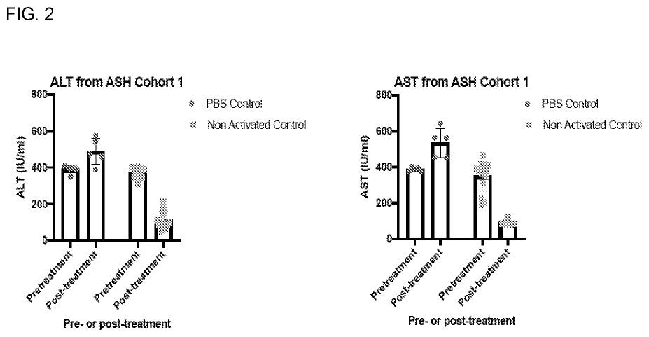

All animals had elevated AST and ALT at randomization, indicating the presence of liver injury. The levels decreased markedly in the MSC-treated animals by sacrifice. However, in the PBS treated animals including the sole survivor, the levels remained high (Table 1, FIG. 2).

### Cohort 2—

Surviving mice were euthanized 25 days after the first treatment. Of the 5 non-activated MSC treated mice, 60% survived. Of the 7 PBS (non-treated mice), only 1/7 or 14% survived. 100% of the 21 mice treated with activated MSC survived.

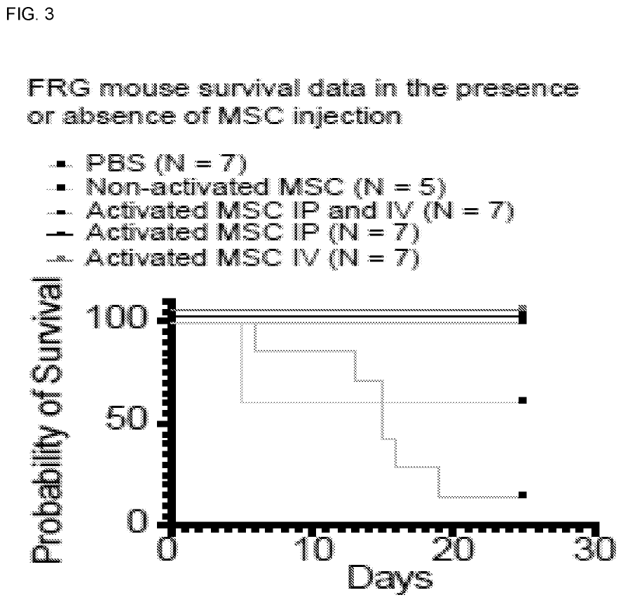

Six of the seven (86%) PBS treated mice died on Days 5-19 after randomization and first treatment. Two of five non-activated MSC treated mice died on Days 4 after randomization and first treatment (FIG. 3). All surviving mice were euthanized 2 days after the final treatment.

A representative section of the liver was fixed in neutral buffered 10% formalin, processed and HE stained sections were obtained and reviewed to evaluate for steatosis, inflammation, necrosis, and fibrosis. Of the 7 placebo mice, 3 showed no significant pathologic changes; 3 showed steatosis, and 2 showed 5-10% necrosis.

Of the 7 mice which received activated mesenchymal stem cells both IP and IV, 5 had 1 steatosis and 2 had no significant findings. No necrosis or significant inflammation was seen in any of the mice.

Of the 7 mice which had activated mesenchymal stem cells IP, 6 had steatosis and 1 had no significant findings. No necrosis or significant inflammation was seen in any of these mice.

Of the 7 mice which had activated mesenchymal cells IV, 5 had steatosis, 2 had no significant findings, and 1 had necrosis.

Of the five mice treated with non-activated stem cells 2 had steatosis, 3 had no significant findings, and 1 had necrosis.

No fibrosis was appreciated in any of the groups on HE stains.

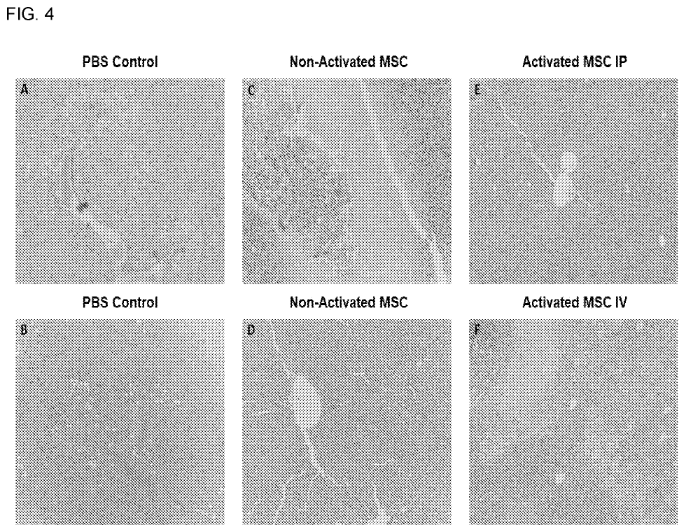

FIG. 4 demonstrates some of the pathology findings at death or at euthanasia.

All of the three activated MSC injection routes rescued alcoholic hepatitis mortality, indicating that IP and/or IV can be utilized for the MSC treatment. Pathological examination showed no significant difference among all the groups, indicating that MSC treatment may have impact on systemic improvement of alcoholic hepatitis.

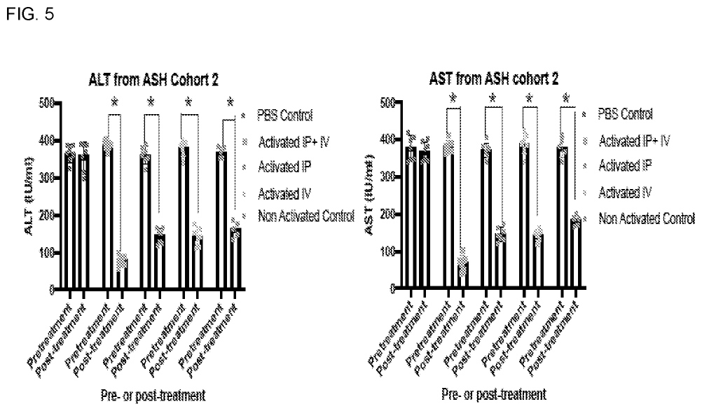

AST and ALT were drawn at onset of treatment and at death including those that were euthanized. All mice had elevated enzymes at the time of randomization indicating liver damage. All PBS treated mice including the one surviving mouse had elevated enzymes at death. All mice receiving non-activated or activated cells including those that died (2 with non-activated cells) demonstrated a significant decrease in the enzymes at time of death. The most pronounced decreases were seen in the mice that received activated cells both IP and IV (FIG. 5).

The MSC group had better survival than the PBS group and the activated MSC group had better survival than the non-activated group further corroborating the role of MSC in survival in this animal model as well as indicating that activated MSC may have better outcomes.

AST and ALT were examined at onset of treatment and at death, including those that were killed. All mice had elevated enzymes at the time of randomization, indicating liver damage. One hundred percent of PBS-treated mice, including the one surviving mouse, had elevated enzymes at death. All mice that received nonactivated or activated cells, including those that died (two with nonactivated cells), demonstrated a significant decrease in the enzymes at time of death. The most pronounced decreases were seen in the mice that received MSCs (p<0.0001) (FIG. 4A,B). To determine the significance of the elevated AST and ALT, a control group of mice was fed isocaloric dextrin-maltose by oral gavage twice a week for 4 weeks without alcohol binging. These mice underwent blood sampling for AST and ALT at the same timepoint as the mice that underwent alcohol binging. ALT and AST levels ranged between 7 U/L and 16 U/L, compared with the elevated labs for the study mice.

### Presence of MSC Lineage Marker Vimentin Validates Human MSCs in Liver

To examine the location of human MSCs, we stained PBS, nonactivated, and activated MSCs with a human-specific MSC lineage marker: Vimentin. Immunohistochemistry revealed Vimentin expression in only activated MSC-treated mice (FIG. 5A). To further analyze Vimentin expression, we isolated RNA from all three mouse groups and performed quantitative polymerase chain reaction (PCR). Quantitative PCR revealed a statistically significant increase of Vimentin expression in activated MSC-treated groups (n=3) compared with PBS controls (n=3) (FIG. 5B). These results validate that MSCs found in the livers of the activated MSC group were in fact human.

K167 and myeloperoxidase complementary DNA levels show the importance of activated MSCs

Ki-67, a liver regeneration marker, has been previously shown to be elevated in patients with alcohol liver. Myeloperoxidase (MPO), a neutrophil marker, has also been shown to be elevated in alcohol-treated mice.

To examine the efficacy of MSCs, we isolated RNA from all three groups of treated mice and performed quantitative PCR (Table S2). Ki-67 expression was significantly elevated in activated MSCs compared with PBS control (FIG. 5C). Conversely, MPO levels were significantly decreased in MSC-treated mice (FIG. 5D). Collectively, these data sets show the importance of activated MSCs in alleviating alcohol-induced liver injury in these mice.

Activated MSC-Treated Mice Retained Human Serum Albumin Levels after Treatment

To examine the quantity of functional hepatocytes in the liver after treatment, we isolated RNA from all three groups and measured human albumin levels relative to mice. Before alcohol liver injury, we showed human mitochondria DNA levels of our humanized FRG mice to be between 60% and 70% (Figure S2). Quantitative PCR analysis revealed a significantly higher human albumin level relative to mouse in activated MSC-treated mice (FIG. 5E). These data indicate the importance of activated MSCs in alleviating liver injury in our humanized mouse model.

### Receptor-Interacting Protein Kinase 3 (RIPK3) Immunofluorescence Shows Ability of MSCs to Inhibit Necroptosis Pathway

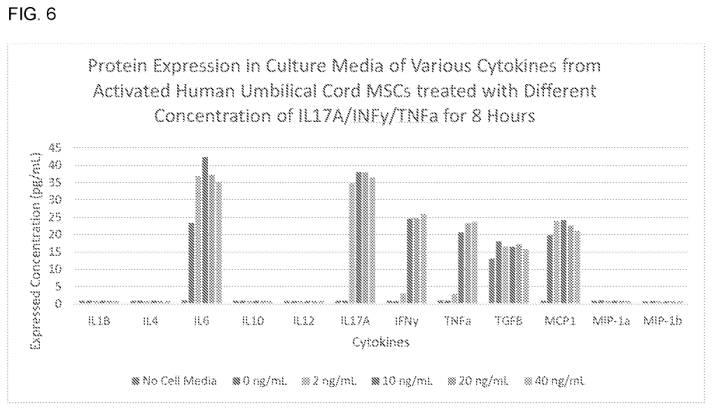

Receptor-interacting protein kinase (RIPK3) has been previously shown to be an important molecule in regulating necroptosis. To determine whether our MSC-treated mice express RIPK3, we stained paraffin-embedded PBS, nonactivated, and activated MSC liver tissue. Confocal microscopy revealed elevated levels of RIPK3 in PBS-treated mice compared with MSC-treated groups (FIG. 6A). The immunoreactive score of confocal images showed significantly lower RIPK3 levels in the activated MSC group compared with PBS control group (FIG. 6B). To confirm the expression of RIPK3 at the protein level, we performed western blot analysis using protein lysates from the livers of activated and PBS treated groups. Our results revealed a decrease of RIPK3 levels in the activated MSC group compared with the PBS control (FIG. 6C). Thus, our RIPK3 studies indicate that the activated MSCs inhibited necroptosis in this mouse group.

### B Cell Lymphoma 2 (BCL2) is Expressed in Activated MSC-Treated Mice

B cell lymphoma 2 (BCL-2) has been well studied as an anti-apoptotic molecule that is involved in necroptosis and pyroptosis pathways. To determine whether BCL-2 is expressed in our alcohol-binged FRG mice, we isolated protein lysates and performed a western blot analysis. Our results revealed BCL-2 expression in activated MSC-treated mice (FIG. 6D). Although BCL-2 was slightly present in the nonactivated MSC-treated group, there was no expression in the PBS-treated mice. These results indicate the importance of activated MSC treatment in alleviating liver injury.

BCL-2 Promoter is Induced after the Addition of MSC Conditioning Media

We next performed luciferase reporter assays targeting signal transducer and activator of transcription 3 (STAT3) and cyclic adenosine monophosphate response element-binding protein (CREB1) in BCL-2 promoter, as BCL-2 has been shown to inhibit necroptosis and pyroptosis. Specifically, Huh7 cells were transfected with various BCL-2 promoter constructs and stimulated with either Plasmalyte or MSC conditioning media. Our results showed that MSC conditioning media turned on BCL-2 expression in the BCL-2 construct with STAT3 and CREB1 deletions (FIG. 6E). Interestingly, other BCL-2 promoter constructs showed minimal relative luciferase activity in both groups. This could be because of the AML-1 (acute myeloid leukemia 1) binding site (−1473 upstream from TSS), which has previously shown to be a repressor of BCL-2 expression.

### Cleaved Gasdermin D Levels Highlight the Importance of Activated MSCs in Alleviating Liver Injury

We next examined Gasdermin D (GSDMD) levels in PBS-treated, nonactivated MSC-treated, and activated MSC-treated groups. GSDMD has been shown to be an important inflammatory response molecule. Western blot showed a reduced expression of cleaved GSDMD in the activated MSC-treated group compared with both nonactivated MSC and PBS control groups (FIG. 6F). This result shows he importance of activated MSCs in alleviating liver injury in our treated mice.

### Transduction of Sh-CD44 Reduces MSCs' Ability to Travel to the Liver

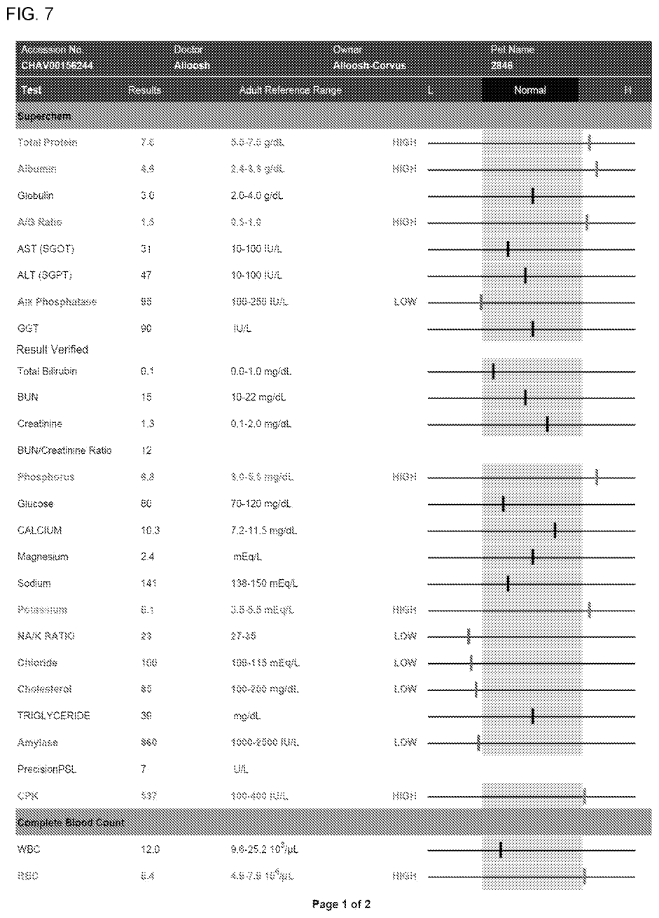

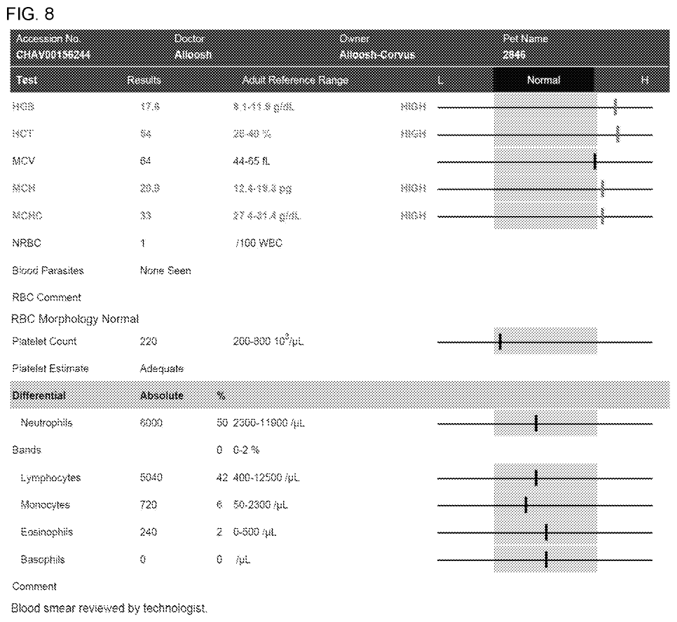

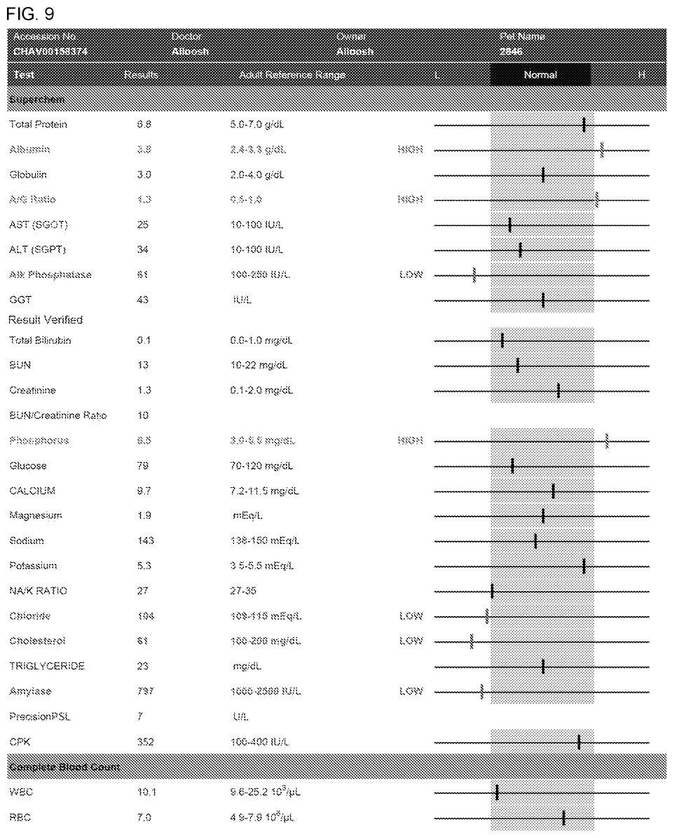

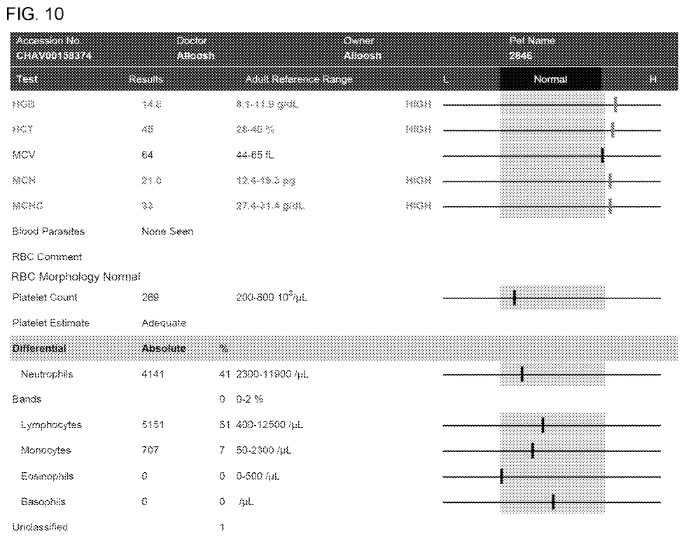

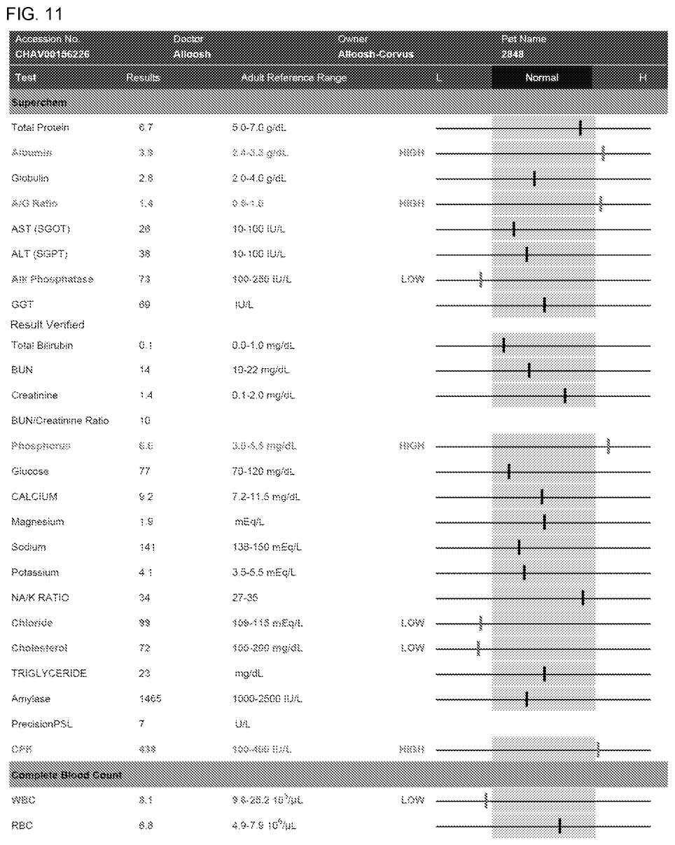

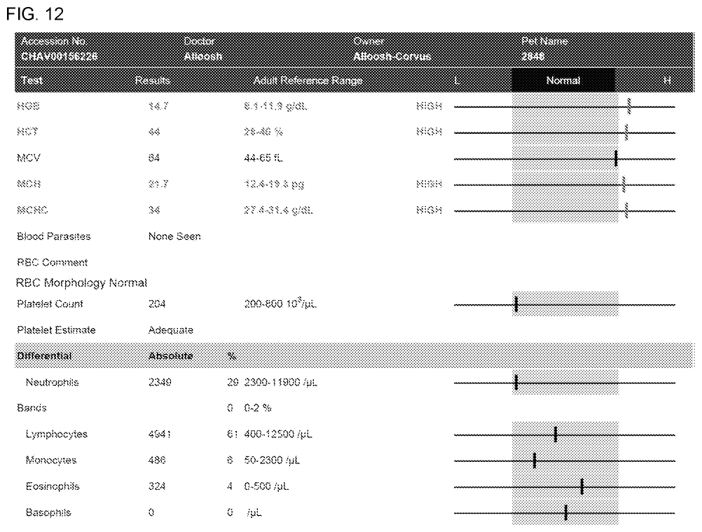

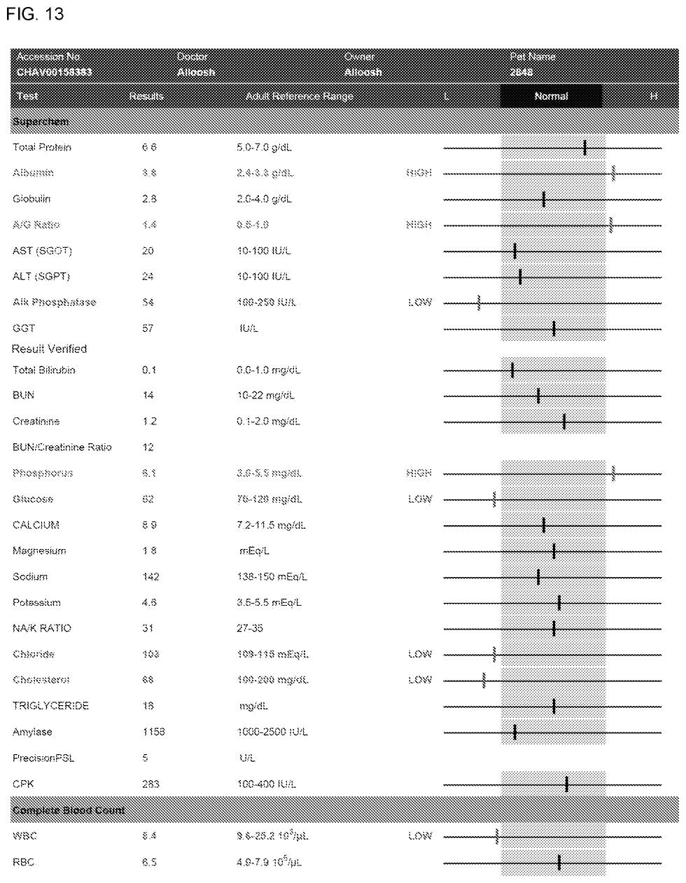

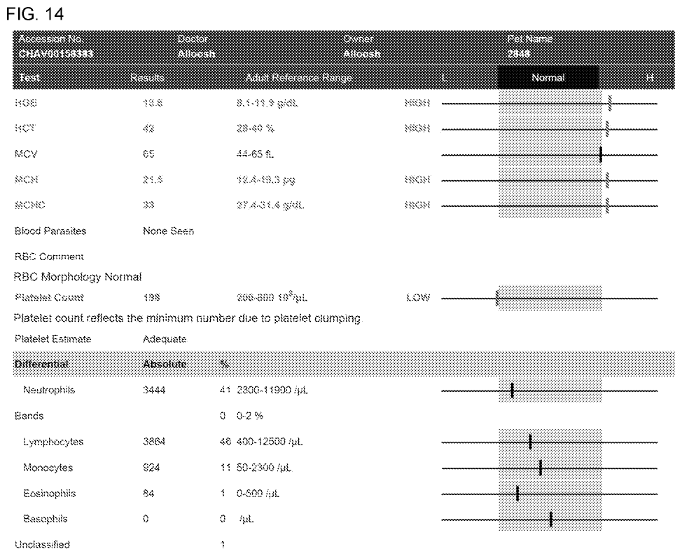

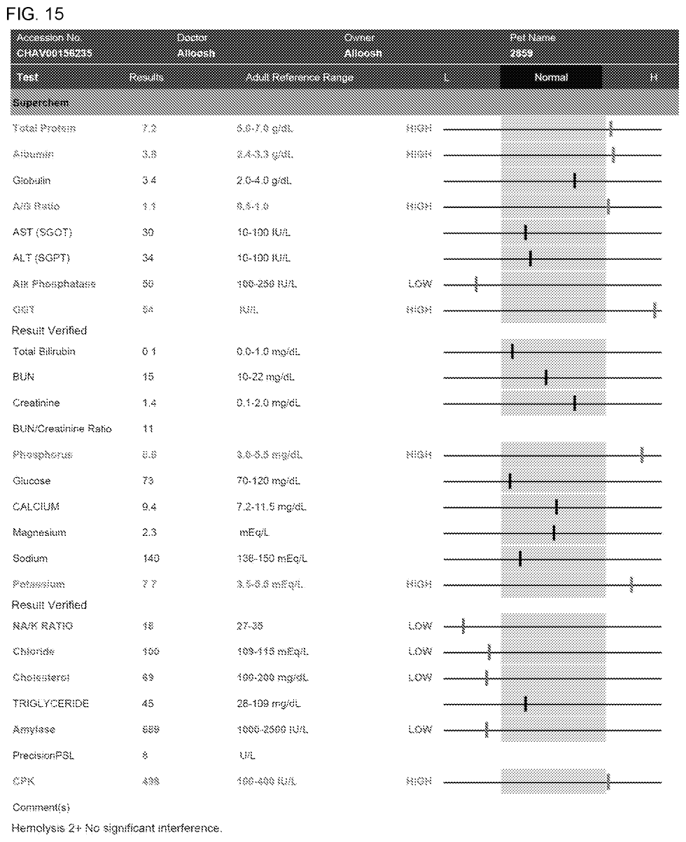

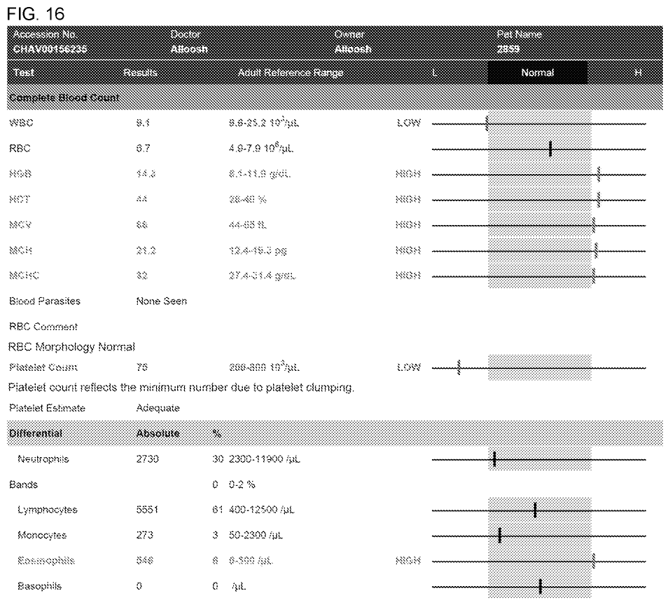

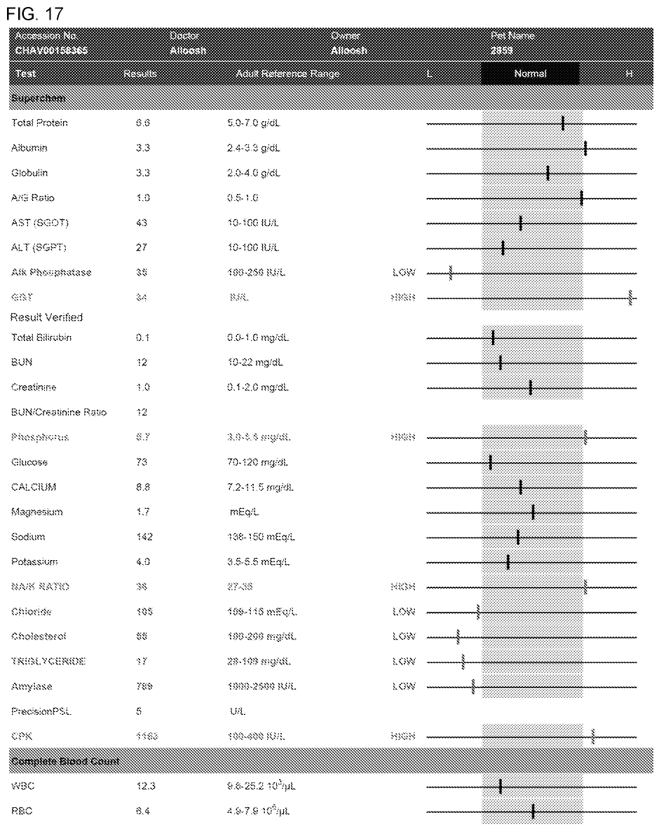

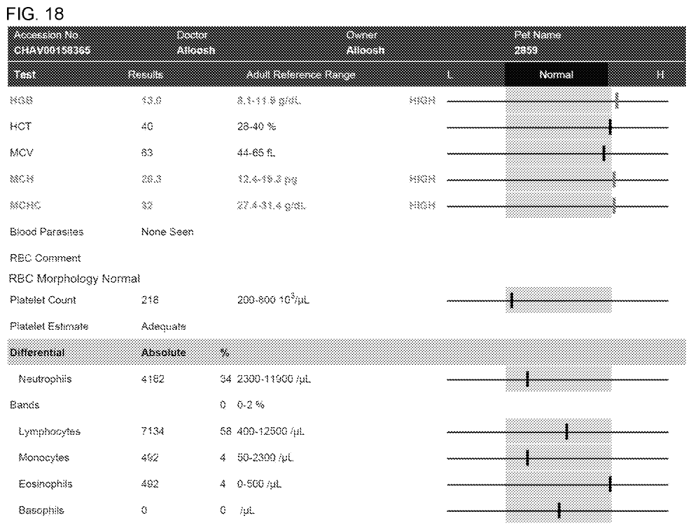

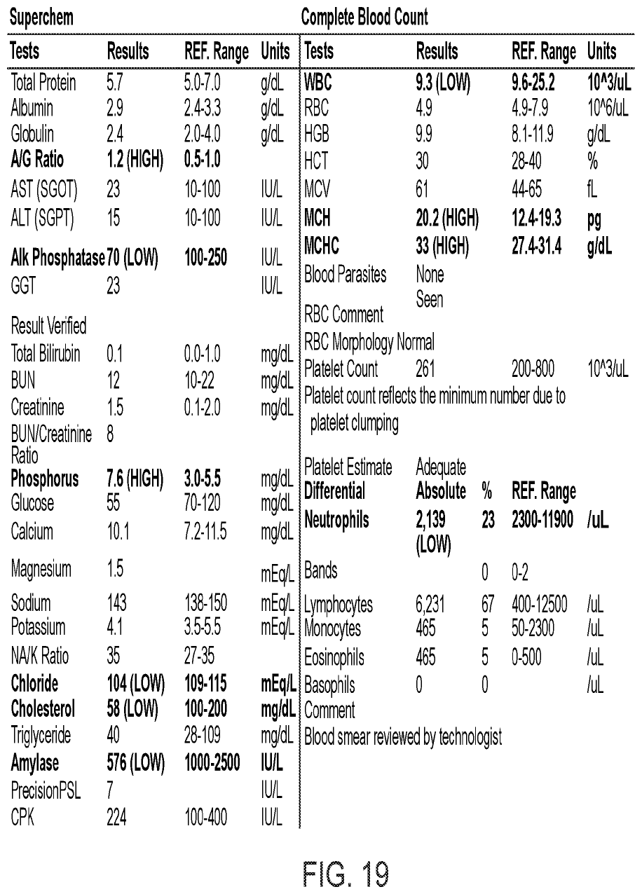

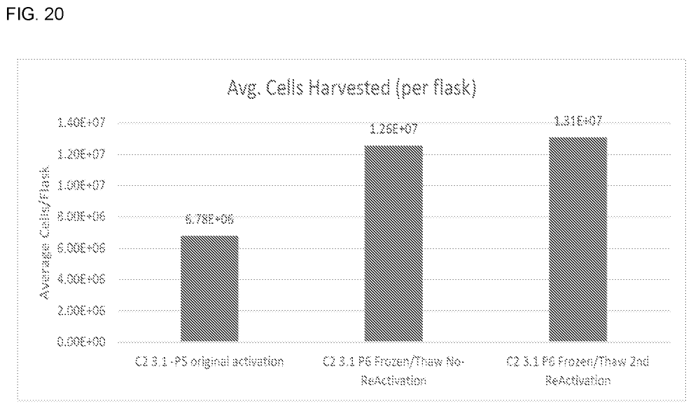

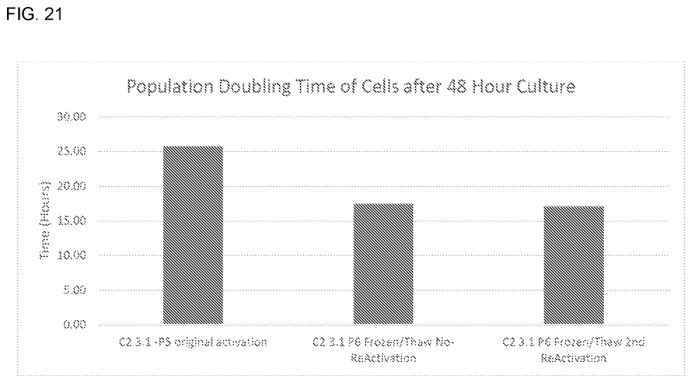

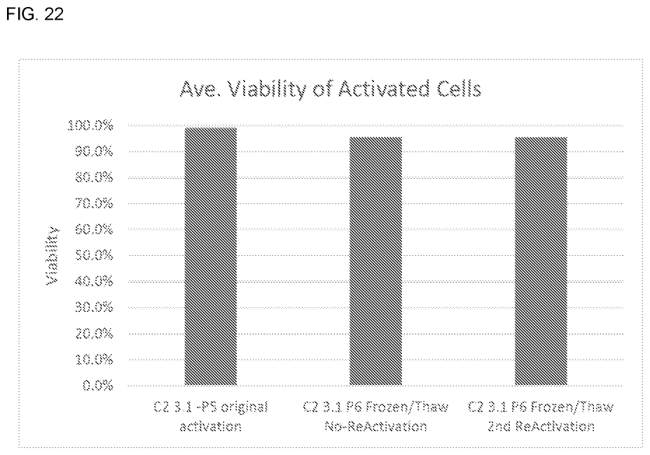

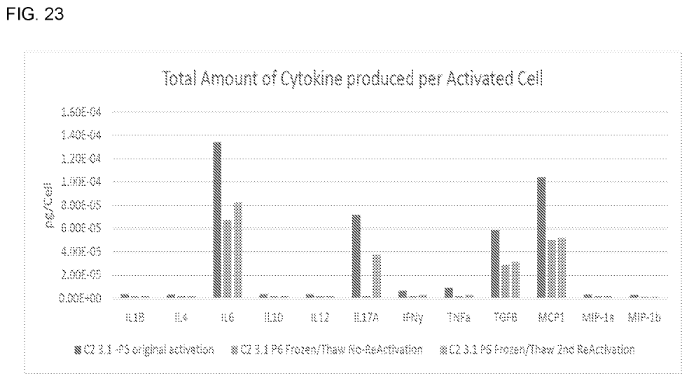

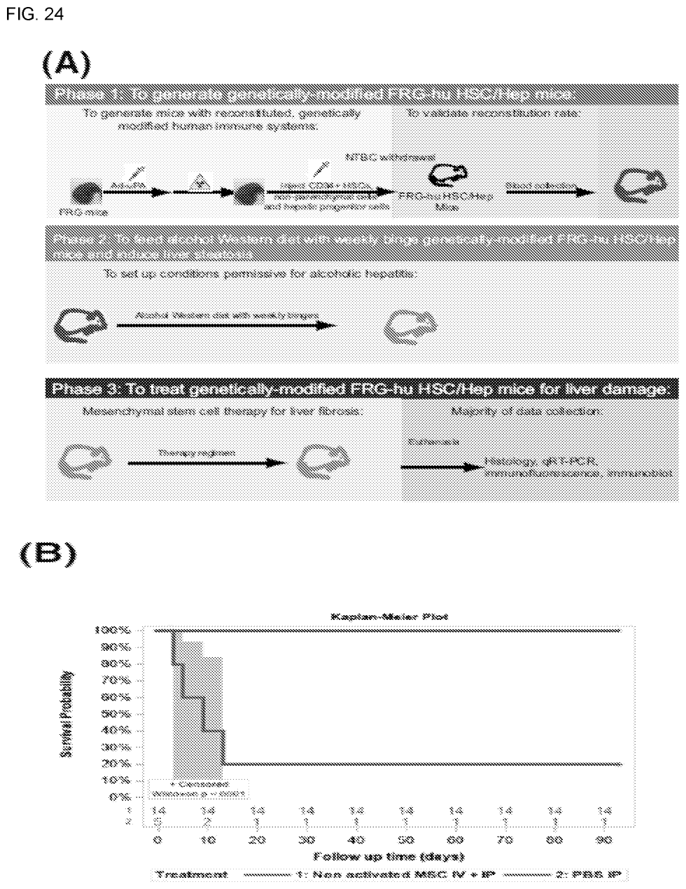

CD44 has been previously shown to be involved in cell trafficking by binding to its ligand hyaluronan. To track the location of where MSCs go after alcohol-induced liver injury, we transduced activated MSCs with sh-CD44 lentivirus. Bioluminescence imaging revealed a higher number of cells present at the liver in sh-scrambled injected mice. In contrast, sh-CD44 injected mice had a lower amount of luciferase expression (FIG. 7A,B). These images show that CD44 has an impact on MSC traveling to the liver after alcohol-induced liver injury.

MSCs have the potential to differentiate into various types of cells, migrate to injured sites, and exhibit anti-inflammatory properties. When tissue damage or injury occurs in the body, MSCs will migrate to the site of injury. Once the MSCs reach this injury site, they interact with various inflammatory cells and different types of stromal cells to start the regeneration process and repair the damaged area. Previous studies have shown that MSCs secrete different types of growth factors, cytokines, and adhesion molecules that affect the damaged tissue area and therefore maintain a positive paracrine effect on the tissue repair process. Other studies have shown that MSCs can produce many different growth factors such as vascular endothelial growth factor, hepatocyte growth factor, epidermal growth factor, fibroblast growth factor, platelet-derived growth factor, insulin-like growth factor 1, and IL-6. Most of these cytokine factors are up-regulated by the activation of NF-κB, from the exposure of pro-inflammatory stimuli such as TNF-α, IFN-γ, IL-1β, lipopolysaccharide, and hypoxia. Several studies propose that MSCs are not spontaneously immunosuppressive but that they require activation for the up-regulation of their immunomodulatory properties. The most important activating or priming factors of MSCs are IFN-γ, TNF-α, IL-17, and IL-1β. After MSC activation from these three pro-inflammatory cytokines, these growth cytokine factors are up-regulated to promote tissue regeneration and repair by the recruitment or stimulation of tissue progenitor cells, fibroblasts, and endothelial cells in the damaged tissue area or by production anti-inflammatory cytokines. These activated MSCs can function to inhibit the proliferation of T helper and cytotoxic T cells through various pathways. The initiation of the anti-inflammatory response is triggered by the activation of T helper type 2 cells and regulatory T cell differentiation. IL-6 can inhibit the maturation of immature dendritic cells and inhibition of T-cell activation by the reduction in the expression of co-stimulatory molecules CD40, CD80 and CD86, by suppression of proinflammatory cytokines and up-regulation of anti-inflammatory cytokines like IL-10. In previous studies, using our proprietary method, our activated MSCs were able to highly express IL-6 in vitro. We propose that the increased production of IL-6 in our activated MSCs could be responsible in modulating the inflammatory conditions in the acute alcoholic liver injury model, to increase survival, prevent apoptosis, and pyrolysis. In future studies, we would like to analyze all of the potential pro-inflammatory and anti-inflammatory our treatment groups to better understand and propose potential anti-inflammatory, anti-apoptosis pathways, and mechanisms to explain why our activated MSCs increased survival in our model. Acute alcoholic hepatitis differs from chronic liver disease in many aspects, most importantly in the potential for reversibility. Therefore, the humanized mouse liver injured with alcohol binging presented the ideal model to test bot nonactivated and activated umbilical cord cells for the potential of increasing survival and affecting the course of the liver injury. Our first alcoholic hepatitis cohort had two groups (PBS control and nonactivated MSC treatment). The nonactivated MSC-treated mice all survived, while the PBS-treated control group had a 20% survival rate. Statistical significance showed p<0.0001. In the second cohort, activated MSC-treated mice had 100% survival, nonactivated MSC-treated mice had 60% survival, and, like cohort 1, the PBS control group had 14% survival.

Analysis of hepatic chemistries before and after treatment with PBS or cells revealed a significant improvement in the animals receiving MSCs compared with those receiving PBS. Furthermore, those receiving activated cells demonstrated more marked improvement compared with nonactivated cells. In cohort 1, there were varying degrees of steatosis in the PBS-treated mice, whereas there were no significant findings in the 14 surviving mice treated with MSCs. We hypothesized that the lack of findings in the surviving mice is likely due to the prolonged time of observation, allowing healing of the liver and corroborated by the marked decrease in hepatic chemistries. In cohort 2, the animals were killed 2 days following the final treatment, and most of the mice showed varying degrees of steatosis as well as other signs of injury. In addition, as the liver pathology did not explain the differences in survival, we can hypothesize that the alcohol may have had a more systematic effect. Publicly available RNA-sequencing data sets have shown the importance of BCL-2 and CD44 in pyroptosis and necroptosis pathways. Our findings with RIPK3, BCL-2, CD44, and GSDMD provide clues on whether activated MSCs alleviated liver injury inhibiting necroptosis and pyroptosis (FIG. 6G). In the future we would like to perform chromatin immunoprecipitation/quantitative PCR and site-directed mutagenesis to see whether CREB1 and STAT3 do in fact turn on BCL-2 promoter. The results in our two cohort experiments show promising results as a treatment to combat alcoholic hepatitis. Although our mice studies were limited in numbers, we look forward to a higher number of FRG mouse cohorts and eventually larger animal studies. It would be interesting to see how long-term high fat/cholesterol+alcohol binge feeding would fare with activated MSC treatment. In summary, activated MSC treatment is a strategic strategy to rescue the high mortality rate of patients with alcoholic hepatitis.

### Example 2—Use of Frozen/Thawed MSC

Following an acute injury, the liver can either regenerate and recover or develop end stage liver failure. The balance between recovery and failure can be impacted by several factors including but not exclusively extent of injury and underlying liver disease. In previously reported studies this group demonstrated that activated umbilical cord Mesenchymal Stem Cells (MSCs) administered to mice with humanized livers who developed liver injury secondary to alcohol, can significantly impact survival.

The primary objective of this study was to evaluate the safety and efficacy of various doses of frozen-thawed activated MSCs compared to placebo in the treatment of acute alcohol induced liver injury in humanized mouse livers. The secondary objectives include evaluation of hepatic chemistries, biomarkers and pathology at various doses.

62 humanized mice that were fed high fat diet and alcohol binge drinking for 24 days were randomized to receive either 1 million, 500,000, 250,000, 100,000, 28,000 activated umbilical cord cells or vehicle (plasmalyte) only injections via tail vein three times in the first week and weekly for two additional weeks. AST and ALT were obtained at baseline, at weeks 1, 2 and 3 and/or at death. Mice were followed for survival at 4 weeks with surviving mice euthanized. Liver pathology was evaluated for all animals at death. Time-to-event data were analyzed using Kaplan Meier curve and log-rank or Wilcoxon rank test, with Sidak method for multiple comparison adjustment, when appropriate. Histology for all mouse livers was reported at time of death.

At the highest administered dose, 1 million stem cells, there was a statistically significant survival compared to the placebo group (p=0.03). Histologic findings correlated with survival with 27 surviving animals demonstrating 1 to 2+ steatosis with no necrosis and 23 of the 35 animals that died demonstrated necrosis with all but 3 of the remaining mice demonstrating various degrees of steatosis.

Treatment with high dose frozen-thawed activated umbilical cord MSCs can result in improved survival and histology in mice with humanized livers and alcohol induced liver injury.

The liver is the main site of alcohol metabolism and has been described as the main target of alcohol-induced injury. The spectrum of liver disease varies from the development of steatosis, steatohepatitis, fibrosis, acute alcoholic hepatitis and advanced liver disease including cirrhosis. Acute alcoholic hepatitis is an inflammatory disease of the liver associated with recent heavy binge drinking and characterized by steatosis, hepatocyte ballooning, Mallory Denk bodies and lobular inflammation including a prominent component of neutrophils. Outcomes are variable with a high 30 day mortality rate for severe cases defined by the discriminant function, reported to be 30-50%. Treatments are primarily supportive with variable reports of efficacy with different therapeutic techniques. Criteria for transplantation are variable between centers and with a limited supply of organs, the need for an effective treatment is imperative.

The remarkable regenerative properties of the liver are influenced by many factors that can change the balance between recovery and failure. The potential of MSCs to promoting regeneration while decreasing the inflammatory response to the liver injury suggest that activated stem cells may offer some advantage in promoting regeneration and improving outcomes in acute liver failure. In previously published work, our group demonstrated improvement in survival in mice with humanized livers that underwent liver injury with binge alcohol drinking using repeated 1 million MSC injections (Table. In addition, survival was significantly improved using activated umbilical cord MSCs compared to non-activated cells and both were significantly better than placebo.

To better determine the optimal dose, evaluate for toxicities at various dose levels and follow up hepatic chemistries and histologic findings, a new set of experiments was designed to compare the various doses, compare these doses to placebo and obtain additional histologic and biochemical data.

In addition to our own prior study, several animal models have demonstrated the ability of MSCs to ameliorate organ failure following liver injury. There have been demonstrations of improved survival, histology, hepatic chemistries and inflammatory markers (Table 6).

### Materials and Methods:

### Preparation of Humanized Mice

After obtaining IACUC approval, we utilized the FRG KO liver-humanized mice. This process is conducted as per routine in the laboratory of Dr. Keigo Machida.

### Breeding of FRG Mice

Our lab generated Fah−/−; Rag2−/−; Il2rgc−/− (FRG) by crossbreeding of Fah−/− mice (RIKEN) and Rag2−/−; Il2rgc−/− (Jackson Lab). These FRG mice are different from commercially available strain. Genotyping was done by following to USC genotyping guideline.

### Irradiation of Newborn FRG Pups

Irradiation was performed at 250 kV, 16 mA, 50 cm FSD using 2 mm filter. Dose exposure rate (cGy): 150 cGy on a field size of 10 cm by 10 cm. Mice were housed in a pathogen-free facility with microisolator cages and monitored to assure there was no acute illness.

### Transplant Procedures

Human hematopoietic stem cells (HSC) were collected from fetal liver (Donor cells). Human HSCs were prepared in sterile media and injected into the irradiated recipients interhepatically. We used 5×10\hat{ }5 cells per mouse, which were injected one day after irradiation procedure.

### Sterilized Water Feeding

We fed animals with autoclaved/irradiated food and maintain them on acidified autoclaved water with or without SMZ (7.8 ml of SMZ per 250 ml of drinking water) on alternate weeks for the duration of their lives after animals are weaned at 3 weeks of age. Animals were monitored daily by the investigator following irradiation and HSC transplant for potential signs of complication (Poor body condition/weight loss, rough coat, inactivity, hunched posture, death without prior signs of illness). Body weight was measured.

### In Vivo Blood Studies of Humanized Immune Cells in FRG Mice

To determine if humanized immune cells are kept in FRG mouse blood stream at the enough levels, blood was collected from the facial vein to detect the human stem cell xenografted mice

FACS Analysis Using Peripheral Blood Cells from FRG Mouse Reconstituted with Human HSCs

Approximately 100 μl of blood per mouse was collected in 1.5-ml sterile microcentrifuge tubes containing 100 μl of 20 mM PBS-EDTA and place it on ice. PBMC's were resuspended the bottom portion (PBMCs) in red blood cell lysis buffer (1×ACK lysis buffer), incubate for 5 min at room temperature (25° C.). The cells were centrifuged at 469 g twice and resuspended in 2% (vol/vol) FBS/PBS containing human CD45, mouse CD45 antibodies and 7-AAD mixture. We measured the human immune reconstitution (% of human CD45+ cells/total CD45+ cells) was examined using flow cytometry analyses.

### Preparation of Umbilical Cord Mesenchymal Cells and Activated Cells

### Human Umbilical Cord-MSC Culture

Human Umbilical cord mesenchymal stromal cells (hUC-MSCs) under informed consent were isolated from the perivascular Wharton's Jelly region of the human umbilical cord were provided by RoosterBio Inc (Frederick, MD; RoosterVial-hUC-XF manufactured and sold by RoosterBio, INC and supported by licensed technology from Tissue Regeneration Therapeutics Inc. (TRT) core technology and patent family: U.S. Pat. Nos. 8,790,923; 8,278,102; 7,547,546; 9,611,456; 9,611,456; 8,481,311; 9,611,456).” The purchased hUC-MSC vials were additionally fully characterized according to the International Society for Cell and Gene Therapy's (ISCT) minimal criteria (24) performed by RB. RoosterBio further performed additional tests for hUC-MSC characterizations for the expression of surface markers by flow cytometry, trilineage mesoderm differentiation potential (adipocytes, osteocytes, and chondrocytes), Indoleamine 2,3-dioxygenase (IDO) activity, sterility, endotoxin, and mycoplasma test (Data not shown). The hUC-MSCs were cultured and harvested following RB manufacturing protocols. One million five hundred thousand to two million cells hUC-MSCs were plated in T225 vented flask (Corning or ThermoFisher) in 25 ml of RB complete medium RoosterNourish-MSC-XF (RoosterBio Inc.) and cultured for 48 hours and incubated at 37° ° C. with 5% CO₂.

Activation of hUC-MSCs

At 36-38 hours after initial culture of hUC-MSCs, Triple activation consisting of human TNF-a (PeproTech, Inc), human INF-g (PeproTech, Inc) and human IL-17 (PeproTech, Inc) was added to each T225 Flask with hUC-MSCs at final concentration of 2 ng/ml for each cytokine (25). Each hUC-MSC Flasks were allowed to culture with added activation media for an additional 10-12 hours in at 37° ° C. with 5% CO2 incubator.

Collection of hUC-MSCs

Cell harvesting from the T225 flasks was performed as follows: Rooster culture medium was removed and washed with 10 mL D-PBS (Gibco) and removed, then added 10 ml of CTS-TrypLE (Gibco) and incubated at 37° C. for 5-6 mins. 10 ml of Rooster media was then added to quench the trypsin activity. Then cell suspension was removed, and T225 Flask was additionally washed with 25 ml D-PBS^{-/-} and placed in centrifuge tube. All the cell suspension mixture was centrifuged at 280×g for 10 min at 4° ° C. and supernatant removed.

Freezing of Activated hUC-MSCs

Pelleted Activated hUC-MSCs was resuspended in 1 ml of CS10 freezing media (BioLife) at a concentration of 5,000,000 or 10,000,000 cells/mL and aliquoted into 1.8 ml freezing vials (Nunc). Aliquoted Cell vials were then frozen using Planer Kryo-550-16 Control Rate Freezer (Planer Limited). Then transferred to Vapor phase LN2 tank for storage until use.

Shipping Cells and Syringe Preparation of hUC-MSCs

One frozen vial of Activated hUC-MSCs was thawed using ThawStar Automated Cell Thawing System (BioLife Solutions). Resuspend thawed frozen cells slowly in 7 ml of Complete Rooster Nourish Media. Frozen-thawed cell suspension tube was centrifuged at 280×g for 10 min at 4° C. Remove supernatant and pelleted Activated hUC-MSCs was resuspended in 10 mL of Rooster Nourish Complete Media. Cell counts were taken using NuceloCounter NC-200 Cell Counter (Chemometec). Cells were aliquoted at a concentration of ˜1,300,000 cells/mL in Complete Rooster Nourish Media into a 1.8 mL vial for each dose (this will ensure that 1,000,000 hUC-MSCs will be injected into each subject). Then, individual Activated hUC-MSCs dose vials were placed into and transported using PrimeGen's proprietary Validated 4° C. Shipping Transportation Box. When ready to use for treatment groups, each vial with activated hUC-MSC suspension was centrifuged at 280×g for 10 min at 4° C. Remove supernatant and pelleted Activated hUC-MSCs were resuspended at a concentration of 1,300,000 cells in 200 uL Plasma-Lyte. The hUC-MSC/Plasma-Lyte solution (200 uL) was loaded into one U-100 BD Ultra-Fine Short Insulin Syringes (Beckton, Dickinson, and Company) for a tail vein injection in mice immediately.

### Binge Drinking

Of the 64 mice that were begun on the binge drinking regimen, 62 survived and were randomized as follows:

### Treatment Groups

The surviving 62 mice were randomized according to sex to one of the following 6 groups:

Group 1: Injected 1 million activated MSCs

Group 2: Injected 500,000 million activated MSCs

Group 3: Injected 250,000 million activated MSCs

Group 4: Injected 100,000 million activated MSCs

Group 5: Injected 28,000 million activated MSCs Group 6: Injected vehicle plasmalyte only

Mice were injected 3 times a week for the first week and then weekly for the remaining 3 weeks. Mice were injected via the tail vein. Each group was assigned 5 male mice and 5 female mice with the exception of the control group assigned 4 females and 6 males. The additional 2 female mice were assigned one each to Group 1 and Group 4

Half of the mice began their first injection on Day 0 following binge drinking and half on Day 1 following binge drinking. The reason for dividing the beginning injection over two days was due to the time required to draw blood and inject the animals and maintain the appropriate documentation. Following the first injection, each group continued injections 3, 7, 14 and 21 days after the first injection and were followed for up to 28 days following the start of the first injections.

Mice were examined during the follow up period for body stance, grooming, respiratory rates, weight and food consumption.

Blood was drawn for AST and ALT prior to each injection and at the time of death.

Necropsy was performed for mice that died before endpoint. Mice with body symptoms were monitored and once their body condition deteriorated to the threshold of euthanasia end point, mice were euthanized based on USC IACUC guideline.

### Pathological Analysis and Evaluation of Liver and Blood of Humanized FRG Mice

We collected blood from FRG-hu HSC/Hep mice to measure human leukocyte reconstitution at 4 weeks post infection and at euthanasia endpoint, 60 days after initiation of MSC treatment (day 167). This blood was collected to measure human transplant efficacy of fetal liver cells. Blood was collected at baseline and at death to measure AST and ALT.

After euthanasia, representative section of liver tissue was fixed with neutral buffered 10% formalin and processed for histological evaluation.

### Statistical Design

Time-to-event data were analyzed using Kaplan Meier curve and log-rank or Wilcoxon rank test, with Sidak method for multiple comparison adjustment, when appropriate.

Kaplan-Meier curve and Wilcoxon rank test were used for data analysis between groups. For post-hoc comparison of each treatment and control group, Sidak adjusted p-value was reported with multiple comparison adjustment.

### Results:

### Group Comparisons:

Tables 4A and 4B demonstrates the age, sex, start date for injections and baseline AST and ALT for all 6 cohorts.

A

| | Total | | Placebo | | Tx 28,000 cells | | Tx 100,000 cells | | Tx 250,000 cells | | Tx 500,000 cells | | Tx 1 million cells | | |
|---|---|---|---|---|---|---|---|---|---|---|---|---|---|---|---|
| | | Median | | Median | | Median | | Median | | Median | | Median | | Median | p- |
| | N | (Q1-Q3) | N | (Q1-Q3) | N | (Q1-Q3) | N | (Q1-Q3) | N | (Q1-Q3) | N | (Q1-Q3) | N | (Q1-Q3) | value |
| Age | 62 | 9 (8- | 10 | 9 (8- | 10 | 9.5 (9- | 11 | 9 (9- | 10 | 8.5 (8- | 10 | 9 (8- | 11 | 9 (8- | 0.72 |
| (weeks) | | 10) | | 10) | | 10) | | 10) | | 10) | | 9) | | 10) | |
| AST at | 62 | 231.5 (188- | 10 | 211 (183- | 10 | 244 (207- | 11 | 234 (194- | 10 | 218 (128- | 10 | 249 (245- | 11 | 169 (120- | 0.13 |
| baseline | | 270) | | 383) | | 311) | | 289) | | 253) | | 336) | | 239) | |
| ALT at | 62 | 288.5 (228- | 10 | 372.5 (291- | 10 | 242 (160- | 11 | 228 (178- | 10 | 270.5 (182- | | 384.5 (355- | | 308 (264- | 0.002 |
| baseline | | 378) | | 401) | | 345) | | 267) | | 338) | 10 | 514) | 11 | 353) | |

B

| | | | | | | | | | Tx | | | | | | |
|---|---|---|---|---|---|---|---|---|---|---|---|---|---|---|---|
| | | | | | | | Tx | | 250,000 | | Tx | | Tx 1 | | |
| | | | | | Tx | | 100,000 | | cells | | 500,000 | | million | | |
| | Total | | Placebo | | 28,000 | | cells | | N | | cells | | cells | | p- |
| | N | % | N | % | N | % | N | % | cells | % | N | % | N | % | value |
| Sex | | | | | | | | | | | | | | | 0.99 |
| F | 33 | 53.2 | 6 | 60 | 5 | 50 | 6 | 54.5 | 5 | 50 | 5 | 50 | 6 | 54.5 | |
| M | 29 | 46.8 | 4 | 40 | 5 | 50 | 5 | 45.5 | 5 | 50 | 5 | 50 | 5 | 45.5 | |
| cohort | | | | | | | | | | | | | | | 0.99 |
| Jun. 11, 2021 | 31 | 50 | 5 | 50 | 5 | 50 | 6 | 54.5 | 5 | 50 | 4 | 40 | 6 | 54.5 | |
| Jun. 12, 2021 | 31 | 50 | 5 | 50 | 5 | 50 | 5 | 45.5 | 5 | 50 | 6 | 60 | 5 | 45.5 | |

Only ALT at baseline was statistically difference between the groups and had no impact on survival.

TABLE 5

| Number of cells per injection | |
|---|---|
| compared to Placebo | Adjusted p-value |
| 28,000 | 0.81 |
| 100,000 | 0.27 |
| 250,000 | 0.18 |
| 500,000 | 0.46 |
| 1 million | 0.03 |

TABLE 6

| Dose | Sur- | | | 1-2 + | 3 + | No |
|---|---|---|---|---|---|---|
| (Activated | vived | | Necro- | Stea- | Stea- | Signif- |
| Stem | to End | | sis | tosis | tosis | icant |
| Cells) | Date | Number | Present | Present | Present | Findings |
| 1 million | No | 3 (27%) | 2 | 0 | 3 | 0 |
| 1 million | Yes | 8 (73%) | 0 | 8 | 0 | 0 |
| 500,000 | No | 6 (60%) | 4 | 2 | 2 | 0 |
| 500,000 | Yes | 4 (40%) | 0 | 4 | 0 | 0 |
| 250,000 | No | 5 (50%) | 3 | 2 | 3 | 0 |
| 250,000 | Yes | 5 (50%) | 0 | 5 | 0 | 0 |
| 100,000 | No | 6 (54%) | 5 | 5 | 0 | 0 |
| 100,000 | Yes | 5 (46%) | 0 | 5 | 0 | 0 |
| 28,000 | No | 8 (80%) | 5 | 2 | 0 | 2 |
| 28,000 | Yes | 2 (20%) | 0 | 2 | 0 | 0 |
| Placebo | No | 7 (70%) | 4 | 5 | 1 | |
| Placebo | Yes | 3 (30%) | 0 | 3 | 0 | 0 |

### Example 3—Treatment of Liver Disease

A 50 year old male suffers from liver disease. He is treated with 1.5×10⁶ activated MSC by injection. The activation process included 12-hour exposure of the MSC to interferon gamma (IFNγ), Tumor Necrosis Factor alpha (TNFα), and interleukin-12 (IL-12).

The patient's symptoms decrease following the treatment.

### Example 4—Treatment of Liver Disease

A 40 year old female suffers from liver disease. She is treated with 1.2×10⁷ activated MSC by injection. The activation process included 10-hour exposure of the MSC to interferon gamma (IFNγ), Tumor Necrosis Factor alpha (TNFα), and interleukin-17 (IL-17).

The patient's symptoms decrease following the treatment.

### Example 5—Treatment of Liver Disease

A 70 year old female suffers from liver disease. She is treated with 1.8×10⁶ activated MSC by injection. The activation process included 6-hour exposure of the MSC to interferon gamma (IFNγ), Tumor Necrosis Factor alpha (TNFα), and interleukin-17 (IL-17).

The patient's symptoms decrease following the treatment.

### Example 6—Treatment of Liver Disease

A 55 year old male suffers from liver disease. He is treated with 2×10⁶ activated MSC by injection. The activation process included 10-hour exposure of the MSC to interferon gamma (IFNγ), Tumor Necrosis Factor alpha (TNFα), and interleukin-17 (IL-17).

The patient's symptoms decrease following the treatment.

### Example 7—Freeze/Thaw of MSC

P5 Cells were prepared, with 2 injections plated at 1.86×10⁶ cells/flask. Extra cells and media from the activated flasks were frozen and storedr. Leftover cells (End of P5 cells) that were already activated and frozen from previous experiment were thawed and cultured in 2 flasks (Plated P6 cells at 1.86×10⁶ cells/flask). 1 Flask was left to grow for 48 hours and the other was reactivated after 38 hours and cultured additionally for 10 hours.

When the 48 hours had elapsed both flasks were harvested and media from each condition was used for the Qiagen Multi-Analyte ELISA Kit.

| | Total | Avg. Cells | Population | Average |
|---|---|---|---|---|
| | Pooled | Harvested | Doubling | Viability |
| | Cells | (per flask) | Time (PDT) | of Cells |
| C2 3.1 original-Frozen (P-5 cells with 1 time activation) | 5.43E+07 | 6.78E+06 | 25.73 | 99.1% |
| C2 3.1 Non-ReActivation-Fresh (P-6 cells that was previously | 1.26E+07 | 1.26E+07 | 17.44 | 95.6% |
| activated frozen thawed and cultured 48 hr) | | | | |
| C2 3.1 ReActivation-Fresh (P-6 cells that was previously activated | 1.31E+07 | 1.31E+07 | 17.07 | 95.6% |
| frozen thawed and reactived a 2^{nd} time and cultured 48 hr) | | | | |

ELISA Results

| | Total Concentration (pg)/Total Cells per Flask = Concentration per Cell | | | | | |
|---|---|---|---|---|---|---|
| | IL1B | IL4 | IL6 | IL10 | IL12 | IL7A |
| C2 3.1 | 3.63E−06 | 3.42E−06 | 1.34E−04 | 3.55E−06 | 3.63E−06 | 7.17E−05 |
| original | | | | | | |
| C2 3.1 No- | 1.92E−06 | 1.74E−06 | 6.71E−05 | 1.88E−06 | 1.94E−06 | 1.818−06 |
| ReActivation | | | | | | |
| C2 3.1 | 1.818−06 | 1.70E−06 | 8.23E−05 | 1.78E−06 | 1.918−06 | 3.76E−05 |
| ReActivation | | | | | | |

| | Total Concentration (pg)/Total Cells per Flask = Concentration per Cell | | | | | |
|---|---|---|---|---|---|---|
| | IFNγ | TNFa | TGFB | MCP1 | MIP-1a | MIP-1b |
| C2 3.1 | 6.23E−06 | 8.936−06 | 5.84E−05 | 3.04E−04 | 3.41E−06 | 2.84E−06 |
| original | | | | | | |
| C2 3.1 No- | 1.90E−06 | 1.88E−06 | 2.89E−05 | 5.01E−05 | 1.84E−06 | 1.49E−06 |
| ReActivation | | | | | | |
| C2 3.1 | 2.91E−06 | 2.96E−06 | 3.14E−05 | 5.20E−05 | 1.69E−06 | 1.48E−06 |
| ReActivation | | | | | | |

This shows growth and viability data of the activated cells when activated once (P5 cells), when activated P5 cells are frozen/thawed and cultured for additional 48 hours (P6 cells), and when activated P5 cells are frozen/thawed, reactivated a 2nd time, and cultured for total of 48 hours.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1) A method for treating liver disease comprising administration of MSC to a patient in need thereof.

2) The method of claim 1 wherein said MSC a comprise activated MSC.

3) The method of claim 2 wherein said activated MSC comprise MSC activated with at least one of interferon gamma (IFNy), Tumor Necrosis Factor alpha (TNFα), and interleukin-17 (IL-17).

4) The method of claim 2 wherein said activated MSC comprise MSC activated with at least two of interferon gamma (IFNy), Tumor Necrosis Factor alpha (TNFα), and interleukin-17 (IL-17).

5) The method of claim 2 wherein said activated MSC comprise MSC activated with interferon gamma (IFNy), Tumor Necrosis Factor alpha (TNFα), and interleukin-17 (IL-17).

6) The method of claim 3 wherein said patient is a mammal.

7) The method of claim 4 wherein said mammal is a human.

8) The method of claim 1, wherein said administration comprises at least one of subcutaneous, intra-articular, intra-lesional, intravenous, intra-peritoneal or intramuscular administration

9) The method of claim 9, wherein said MSC are autologous.

10) The method of claim 8, wherein said MSC are allogenic.

11) The method of claim 9 or 10 wherein said MSC are administered in a dose between 1×10³ cells and 1×10¹² cells.

12) The method of claim 11, wherein said dose comprises at least two doses.

13) The method of claim 12, wherein said dose comprises at least three doses.

14) The method of claim 13, wherein said dose comprises at least four doses.

15) The method of claim 14, wherein said dose comprises at least five doses.

16) A method of activating a cytokine-producing MSC, comprising stimulating the MSC with at least one of interferon gamma (IFNy), Tumor Necrosis Factor alpha (TNFα), and interleukin-17 (IL-17).

17) A method of activating a cytokine-producing MSC, comprising stimulating the MSC with at least two of interferon gamma (IFNy), Tumor Necrosis Factor alpha (TNFα), and interleukin-17 (IL-17).

18) A method of activating a cytokine-producing MSC, comprising stimulating the MSC with interferon gamma (IFNy), Tumor Necrosis Factor alpha (TNFα), and interleukin-17 (IL-17).

19) The method of any of claims 16-18, wherein said cytokine comprises at least one of IL-6, IL-17A, IFN gamma, TNFα, TGFβ, MCP1, HGF, IL-8, TIMP-1, TIMP-2, VEGF, IDO, MIP-1b, and IL-10.


SEQUENCE LISTING (10 sequences)

SEQ ID NO: 1 - DNA, 20 bp, Artificial Sequence
  Human albumin sense primer
gaaagcccactgtcttagtg

SEQ ID NO: 2 - DNA, 20 bp, Artificial Sequence
  Human albumin anti-sense primer
gggtgtagcgaactagaatg

SEQ ID NO: 3 - DNA, 18 bp, Artificial Sequence
  Mouse albumin sense primer
gcaaggctgctgacaagg

SEQ ID NO: 4 - DNA, 22 bp, Artificial Sequence
  Mouse albumin anti-sense primer
ggcgtctttgcatctagtgaca

SEQ ID NO: 5 - DNA, 25 bp, Artificial Sequence
  Ki-67 sense primer
tcctttggtgggcacctaagacctg

SEQ ID NO: 6 - DNA, 25 bp, Artificial Sequence
  Ki-67 anti-sense primer
tgatggttgaggtcgttccttgatg

SEQ ID NO: 7 - DNA, 20 bp, Artificial Sequence
  MPO sense primer
ccaacaacatcgacatctgg

SEQ ID NO: 8 - DNA, 20 bp, Artificial Sequence
  MPO anti-sense primer
gctgaacacaccctccttct

SEQ ID NO: 9 - DNA, 21 bp, Artificial Sequence
  Vimentin sense primer
ccaccaggtccgtgtcctcgt

SEQ ID NO: 10 - DNA, 20 bp, Artificial Sequence
  Vimentin anti-sense primer
cgctgcccaggctgtaggtg